(19) United States
(12) Patent Application Publication
(10) Pub. No.: US 2018/0372670 A1
(43) Pub. Date: Dec. 27, 2018

(54) TEST DEVICE AND METHOD OF USING A TEST DEVICE

(71) Applicant: Inside Biometrics Limited, Edinburgh (GB)

(72) Inventors: James Rodgers, Edinburgh (GB); Damian Baskeyfield, Edinburgh (GB); James Moffat, Edinburgh (GB)

(21) Appl. No.: 15/737,801

(22) PCT Filed: Jun. 23, 2016
(86) PCT No.: PCT/GB2016/051888

§ 371 Date: Dec. 19, 2017

(30) Foreign Application Priority Data
Jun. 26, 2015 (GB) 1511299.8

(51) Int. Cl. G01N 27/327; G01N 33/487

(52) U.S. Cl. CPC G01N 27/3274; G01N 33/48771; G01N 27/3272

(57) ABSTRACT

An electrochemical test device for detecting a first analyte and a second analyte in a fluid sample is provided. The device comprises a first conductor layer arranged to receive a fluid sample, wherein the first conductor layer comprises a first working electrode for receiving sensing chemistry for the first analyte and a second working electrode for receiving sensing chemistry for the second analyte; and wherein a layer of the electrochemical test device is arranged to provide error correction information for correcting a measurement of a response of the fluid sample with at least one sensing chemistry received on the first conductor layer.

### FIELD OF THE INVENTION

The present invention relates to a test device for measuring amounts of a plurality of analytes in a fluid sample. The invention further relates to a method of using such a test device.

### BACKGROUND TO THE INVENTION

The detection and measurement of substances, chemicals, or analytes in a bodily fluid sample is useful in a variety of applications, such as in fitness monitors or in the medical device industry. For example, an individual may choose to monitor a concentration of an analyte such as glycerol and/or lactate in his or her bloodstream in order to determine whether or not a chosen fitness regime is effective. Glycerol is a fitness related analyte associated with lipolysis and fat breakdown from stored body fat.

As another example, people with diabetes need to regularly monitor the concentration of glucose in their bloodstream in order to determine if they are in need of glucose or insulin or other diabetes medication. Diagnostic devices and kits have been developed over the years to allow a diabetic individual to autonomously determine the concentration of glucose in their bloodstream, in order to better anticipate the onset of hyperglycaemia or hypoglycaemia and take any necessary action.

When trying to ascertain a level of an analyte in, for example, a blood sample, an individual will typically perform a finger stick using a lancing device to extract a small drop of blood from a finger or alternative site. An electrochemical test device, which is often a test strip, is then inserted into a diagnostic meter, and the sample is applied to the test strip. Through capillary action, the sample flows through a capillary channel across a measurement chamber of the device and into contact with one or more electrodes or conductive elements coated with sensing chemistry for interacting with a particular analyte or other specific chemical (for example glucose) in the blood sample. The magnitude of the reaction is dependent on the concentration of the analyte in the blood sample. The diagnostic meter may detect the current generated by the reaction of the sensing chemistry with the analyte, and the result can be displayed to the individual.

Typically, such electrochemical test devices have a set of electrodes such as a counter/reference electrode and one or more working electrodes. Sensing chemistry is used which is typically tailored to the particular analyte or biometric of interest. An enzymatic electrode is a combination of an enzyme and an electrochemical transducer. The direct transfer of electrons between the enzyme and the electrode is generally not easy to achieve and so an electron transfer agent (or mediator) is sometimes used to transfer electrons between the enzyme and the electrode to facilitate the electrocatalysis. For example, when measuring the concentration of glucose in a sample, a glucose oxidase or a glucose dehydrogenase enzyme can be used in conjunction with a mediator such as potassium ferricyanide. When detecting other analytes, different enzymes may be used, such as β-hydroxybutyrate dehydrogenase for measuring the ketone body β-hydroxybutyrate.

A significant drawback of testing with such electrochemical test devices is the need for users to lance their fingers to obtain a sample of blood every time a measurement is performed. The pain and inconvenience associated with this step is highly dependent upon the volume of sample required to conduct a test. Systems with smaller sample sizes involve less pain and enjoy greater user acceptance. In the context of self-monitoring blood glucose test strips, test strips capable of measuring a concentration of a single analyte in a fluid sample volume of around 0.3-0.7 μL have been developed.

Additionally, it is important that the reading output by a meter can be relied upon so that, if necessary, appropriate action may be taken. If the reading is erroneous and the user acts upon the erroneous reading, any action taken (e.g. the administration of insulin or sugar) could be detrimental to the user's health. Erroneous readings can arise not only if the test strip is damaged (which could affect the flow of the fluid sample across the measurement chamber) or if the meter itself is damaged, but also if other components of the fluid sample affect the output reading of the meter.

One notable component of a blood sample which may affect the reading output by a meter is the quantity of red blood cells present. This is termed the haematocrit level, also known as packed cell volume (PCV) or erythrocyte volume fraction (EVF). It denotes the volume percentage of red blood cells in any given sample of blood. The haematocrit level heavily influences the diffusion processes underpinning the electrochemical reactions taking place within the test strip, from which the glucose concentration measurement is ultimately generated. In the absence of appropriate and effective haematocrit correction or mitigation strategies, such haematocrit variations can lead to significant systematic biases in the measured results. Typically the mean haematocrit is around 45% for men and around 40% for women. If, for example the haematocrit is higher than this mean (i.e. there are more red blood cells in the fluid sample than typically the case) then it is likely that the measured concentration of the analyte under study will read lower than the actual concentration value. If the haematocrit is lower than the mean (i.e. the red blood cell count in the sample is lower than typically the case) then it is likely that the measured concentration of the analyte under study in the sample will read higher than the actual concentration value.

A number of haematocrit mitigation strategies have been developed and successfully practiced to overcome this limitation in electrochemical test devices which measure the concentration of only a single analyte. However, these haematocrit mitigation strategies can also suffer from some drawbacks. For example, some of the most widespread haematocrit mitigation strategies necessitate additional separate electrodes. This further increases the required sample volume and meter complexity. Some approaches require that a minimum degree of physical electrode separation be maintained between analyte measuring electrodes (such that corrective measurements of properties of the sample fluid which are reflective of the sample haematocrit but independent of the target analyte concentration can be accomplished with sufficient certainty.) This can also increase the required sample volume.

Some approaches necessitate a very high level of control and consistency of the sample capillary dimensions. In practice, this can also lead to a requirement for larger sample volumes, because of the need for these tight manufacturing tolerances to be accommodated in a cost effective manner.

This situation has created an element of ‘trade off’ between the minimum sample volume that is practically achievable within an electrochemical test device, and the simultaneous need to maintain acceptable levels of accuracy across the full range of haematocrit which is observed. Many existing haematocrit mitigation strategies that are capable of meeting the required system accuracy requirements also introduce additional cost and complexity to the measurement system, for example in terms of additional meter functionality and components, or in terms of additional test strip features, and sometimes both. These additional features may in turn entail additional manufacturing steps and/or raw materials and/or acceptance tolerances, with the attendant associated costs therein. For example, additional membrane layers for the exclusion of erythrocytes from the electrode region add cost and manufacturing complexity to the test strip. Additional haematocrit compensation electrode elements add cost and complexity to the test strip.

For users that wish to measure the concentrations of multiple analytes in a fluid sample, these problems are typically amplified. In order to take a first measurement of, for example, a concentration of glucose in the fluid, and then take a second measurement of, for example, a blood ketone in the fluid, often two different electrochemical test devices are required, each with their own specific sensing chemistry for detecting a respective analyte. Accordingly, a user will typically have to provide two fluid samples, leading to increased discomfort.

While multi-analyte test strips are known, these have hitherto suffered from various inherent drawbacks which have rendered them unable to compete directly with today's single analyte electrochemical test devices. For multi-analyte test strips to be able to compete with today's single analyte test strips in respect of user comfort, and accuracy, a number of challenges must first be overcome.

Firstly, the sample size should be as small as those required by modern single analyte devices. A simple juxtaposition of, for example, two single analyte test systems within a common fluidic sampling architecture would result in an approximate doubling of the required sample volume over that for an equivalent accuracy single analyte measurement system.

Secondly, when performing multi-analyte measurements on a single fluid sample, the potential exists for a cross interference between the two analyte readings, such that measurement of the concentration of a first analyte in the sample confounds the measurement of the concentration of a second analyte in the sample and vice versa.

Thirdly, due to the additional complexities described above, haematocrit mitigation strategies that readily lend themselves to a single analyte electrochemical test device do not operate as readily or effectively within the multi-analyte situation. For example, haematocrit mitigation strategies that rely on the measurement of the impedance of the sample between electrodes are sensitive to the composition, solvation and dissolution of any reagent layers being placed upon or between the electrodes by which the impedance is measured.

The present invention seeks to provide an improved multi-analyte electrochemical test device for determining a concentration of one or more analytes in a fluid sample.

### SUMMARY OF THE INVENTION

In a first aspect of the invention, there is provided an electrochemical test device for detecting a first analyte and a second analyte in a fluid sample. The electrochemical test device comprises a first conductor layer arranged to receive a fluid sample, wherein the first conductor layer comprises a first working electrode for receiving sensing chemistry for the first analyte and a second working electrode for receiving sensing chemistry for the second analyte. A layer of the electrochemical test device is arranged to provide error correction information for correcting a measurement of a response of the fluid sample with at least one sensing chemistry received on the first conductor layer.

The first analyte may be glucose and the second analyte may be a ketone body. In some embodiments, the first and second analytes may be the same analyte. The second analyte may be β-hydroxybutyrate.

The electrochemical test device may further comprise a spacer layer defining a sample introduction chamber for receiving input of the fluid sample into the electrochemical test device. The sample introduction chamber may have an upper volume of about 1.0 μL, optionally about 0.7 μL, further optionally about 0.45 μL, and still further optionally about 0.30 μL.

The first working electrode may have thereon first sensing chemistry for the first analyte and the second working electrode may have thereon second sensing chemistry for the second analyte. The first sensing chemistry may comprise a first analyte reagent formulated to react with the first analyte to generate a signal indicative of an amount of the first analyte in the fluid sample, the first analyte reagent having a first time-based response characteristic. The sensing chemistry for the second analyte may comprise a second analyte reagent formulated to react with the second analyte to generate a signal indicative of an amount of the second analyte in the fluid sample, the second analyte reagent having a second time-based response characteristic.

For example, the first and second analyte reagents may solvate differently when brought into contact with the sample, thereby producing analyte-dependent signals with different current vs time characteristics. Relatively fast solvating reagents are more precise with respect to eliminating effects of manufacturing variation but more susceptible to systematic sample errors. Where the sample to be tested is blood, a systematic sample error may be haematocrit. On the other hand, relatively slower solvating reagents are less precise with respect to effects of manufacturing variations but less susceptible to systematic sample errors. For example, a fast solvating reagent would equilibrate within about 10% of the total test time (e.g. the period during which currents at the electrodes are measured), whereas slower solvating reagents would not have fully equilibrated within about >20% of the total test time. The total test time may be of the order of 5-15 s typically, but other test times may be used. The time based response characteristic for each electrode can be established by taking signal readings, for example current readings, at one or more time points within the course of the measurement, and preferably taking readings from multiple time points from within the test, as the reagent pad progresses through different physical stages (e.g. hydration, swelling, solvation, dissolution, equilibration etc.), while the underlying signal generating chemical reactions are proceeding.

The time-based response characteristics may be a function of the respective thicknesses of the first and second analyte reagents. The thicknesses may be defined by the number of layer(s) forming the respective analyte reagents.

The first and second time-based response characteristics may comprise respective different diffusional and/or dissolutional response characteristics. The first analyte reagent may be formulated to more rapidly dissolve and/or diffuse into the fluid sample relative to the second analyte reagent. The first and second analyte reagents may comprise film-forming components and non-film-forming components. The first analyte reagent may comprise a relatively high proportion of non-film-forming components to film-forming components relative to the proportion of non-film-forming components to film-forming components comprised in the second analyte reagent.

The relative rates of reagent layer solvation and diffusion may affect the performance of the sensors described herein, with respect to both effecting appropriate haematocrit mitigations strategies and with respect to the avoidance of crosstalk effects. In designing the reagent layers, appropriate reagent formulation strategies may ensure that the desired dissolution and diffusional characteristics can be achieved. Several strategies for controlling the relative solvation and diffusion rate of the different reagent layers may be employed, such as those described in GB1419799.0, filed 6 Nov. 2014, incorporated herein by reference in its entirety.

For example, a high proportion of non-film-forming soluble components relative to film-forming soluble components can provide for a reagent composition that dries with a high surface area and non-uniform microstructure that dissolves rapidly and permits rapid diffusion. Conversely, a high proportion of film-forming components relative to non-film-forming components can provide a for reagent composition that dissolves more progressively and constrains rapid diffusion for longer. The creation of reagent formulation that dissolve to different extents within different time periods is within the ambit of the skilled person.

Film-forming components include any material that, when cast from solution onto a surface and then dried, exhibits a tendency to form a layer which effectively minimises the surface area at the solid/air interface in order to correspondingly minimise the free surface energy thereat, producing a tendency towards flat and even film layers. An example of one such material is hydroxyethycellulose (HEC), but it will be apparent that other hydrophilic film forming polymers exist which would work as suitable replacements, such as polyvinyl alcohol (PVA), polyethylene oxide (PEO), pullulan, carboxymethylcellulose (CMC), acrylates, water soluble polyesters and polyols, dextrans, xantham gums and similar materials. Preferably, the film-forming components have the following properties: aqueous solubility, viscosity at the desired concentration (with regard to coating performance), and compatibility with other components of the analyte reagent. Higher molecular weight/viscosity grades of polymer can be utilised at lower levels to maintain similar wet reagent behaviour whilst reducing the overall amount of film-forming component present relative to other components. Conversely, lower viscosity/molecular weight analogues at higher levels can be utilised to maintain a suitable coating viscosity for the analyte reagent whilst increasing the ratio of film-forming component to other components.

Thus strategies for modulating the levels of film-forming component relative to other components, while maintaining similar printability behaviour, become apparent to those skilled in the art, as means by which the solvation properties of the analyte reagent layer may be controlled to within desirable limits. Similarly, different polymers possessing greater or less solubility can be used as a means of affecting the solvation properties of the analyte reagent.

Non-film forming components include crystallising materials that disrupt film formation. The processes of crystal formation, for example, can thermodynamically predominate over the minimisation of surface energy at the resulting solid air interface, such that domains of crystal growth extend within and protrude from the film surface, increasing the surface area of the solid/air interface well beyond its minimum, and wherein compositional differences between such crystalline domains and adjacent domains (containing other components) can arise spontaneously through the thermodynamically driven association of like constituents. Examples of rapidly solvating crystallising material include inorganic salts such as potassium phosphate, sodium chloride, potassium chloride, nitrate salts, ammonium salts, organic materials such as sucrose, citrates, ethane-sulfonic-acid salts such as PIPES, HEPES, MES, or any other rapidly solvating crystalline material. The electrochemical transfer agent or mediator may also act as a non-film-forming component.

Alternate means of interfering with the relative film forming vs non film forming propensity of the reagent layer might also be employed, for instance, the incorporation of polymer combinations which exhibit limited compatibility with each, or volatile non-solvents (for example octanol) which disrupt the compatibility of the reagent layer subcomponents during the drying and film forming processes.

The incorporation of surfactant and the level at which it is employed can also influence the wettability of the film and the penetration of sample components there into, influencing the solvation properties. Therefore using different surfactants and/or surfactant combinations to speed up or slow down penetration of the sample into the reagent will be recognised as a viable strategy by which the analyte reagent solvation properties might also be influenced. Those skilled in the art can select a suitable surfactant. Examples include Tergitol NP-9, Triton™ X100; Borchi Gol™LA200, 1375, LA6; Brij™35; Pluronic™P103; Tergitol™ NP-7.

The electrochemical test device may further comprise a third working electrode having thereon a third analyte reagent formulated to react with the first analyte to generate a signal indicative of an amount of the first analyte in the fluid sample, the third analyte reagent having a third time-based response characteristic. The first analyte reagent may be formulated to more rapidly dissolve and/or diffuse into the fluid sample relative to the third analyte reagent.

The electrochemical test device may further comprise a second conductor layer. The second conductor layer may be arranged to provide error correction information for correcting a measurement of a response of the fluid sample with at least one sensing chemistry received on the first conductor layer. In this embodiment, the first and second analytes may be the same analyte. The second conductor layer may comprise an electrode for detecting a volumetric sufficiency of the sample-receiving chamber (generally referred to as fill-sufficiency detect electrode). In the context of the invention, error correction information may be any information that may be used to mitigate or reduce the effect on measurements due to haematocrit, diffusion-related effects, errors caused by other constituents in the fluid sample such as lipids, cholesterols, etc.

The second conductor layer may comprise a first electrode and a second electrode. The first electrode and the second electrode may be spatially separated to form a gap arranged to receive a fluid sample. The spacer layer is disposed between the first conductor layer and the second conductor layer.

The first electrode of the second conductor layer may comprise a first through-hole and the second electrode of the second conductor layer may comprise a second through-hole. A first end of the first through-hole may define a first area on the first working electrode for receiving sensing chemistry for the first analyte. A first end of the second through-hole may define a second area on the second working electrode for receiving sensing chemistry for the second analyte.

The electrochemical test device may further comprise an insulator layer disposed between the first conductor layer and the second conductor layer. The insulator layer may comprise a dielectric material.

The insulator layer may comprise a third through-hole and a fourth through-hole. The third through-hole may fluidly connect the first through-hole to the first area and the fourth through-hole may fluidly connect the second through-hole to the second area.

The through-holes may each have a diameter of about 700 μm.

The sample introduction chamber may be fluidly-connected with the first through-hole and the second through-hole.

The internal volume of the sample introduction chamber, first, second, third and fourth through-holes may be about 0.5 μl.

The electrochemical test device may further comprise a cover layer above the spacer layer for covering the top of the sample introduction chamber.

The fluid sample may comprise one of: blood; plasma; urine; saliva; lacrimal fluid; sweat; interstitial fluid; or breath condensate.

The first conductor layer may further comprise a counter/reference electrode.

The sensing chemistry for the first analyte may comprise a dehydrogenase. The dehydrogenase may be an NAD(P)⁺-dependent dehydrogenase and the sensing chemistry for the first analyte may further comprise a cofactor for the NAD(P)⁺-dependent dehydrogenase.

The sensing chemistry for the first analyte may further comprise a diaphorase and an electron transfer agent.

At least one of the sensing chemistry for the first analyte and the sensing chemistry for the second analyte may comprise an oxidase.

In a further aspect of the invention there is provided an apparatus configured to detect a first analyte and a second analyte in a fluid sample applied to an electrochemical test device according to any of the above embodiments.

In a further aspect of the invention there is provided a method of making an electrochemical test device for measuring the amounts of first and second analytes in a fluid sample. The method comprises applying to a first electrode a first analyte reagent having a first time-based response characteristic and being formulated to react with a first analyte to generate a signal indicative of an amount of the first analyte in the fluid sample. The method further comprises applying to a second electrode a second analyte reagent having a second time-based response characteristic and being formulated to react with a second analyte to generate a signal indicative of an amount of the second analyte in the fluid sample.

There is provided a method of using an electrochemical test device according to any of the above embodiments. The method may comprise using a plurality of electrochemical test devices. The method comprises polarising the first and second working electrodes of each of the plurality of the electrochemical test devices, applying a respective reference blood samples to each of the plurality of electrochemical test devices, each reference blood sample having known amounts of the first and second analytes and a known haematocrit, and, for each of the plurality of reference blood samples, measuring one or more currents generated at each of the first and second working electrodes of the respective electrochemical test device.

The method may further comprise reversing the polarisation. The method may further comprise measuring one or more further currents generated at each of the first and second working electrodes. The method may further comprise determining, based on the one or more measured currents, sensitivity data indicative of a sensitivity of each of the first and second analyte reagents to: the first analyte, the second analyte and the haematocrit of the reference blood sample. The method may further comprise analysing the sensitivity data to determine a correction algorithm. The correction algorithm may compensate for the sensitivity of the first and second analyte reagents to haematocrit. The method may further comprise applying a test blood sample to the test device, measuring one or more currents generated at each of the first and second working electrodes, correcting the one or more measured currents using the correction algorithm, determining an amount of the first analyte and an amount of the second analyte in the test blood sample, based on the corrected one or more measured currents.

In a further aspect of the invention, there is provided a method of manufacturing an electrochemical test device. The method comprises providing a first conductor layer, wherein the first conductor layer comprises a first working electrode for receiving sensing chemistry for the first analyte and a second working electrode for receiving sensing chemistry for the second analyte. The method further comprises providing a layer arranged to provide error correction information for correcting a measurement of a response of the fluid sample with at least one sensing chemistry received on the first conductor layer.

The method may further comprise providing an insulator layer between the first conductor layer and the second conductor layer.

The method may further comprise providing a spacer layer between the first conductor layer and the second conductor layer, the spacer layer defining a sample introduction chamber for introducing a fluid sample to the working electrodes.

In a further aspect of the invention, there is provided a method of using an electrochemical test device according any of the above-described embodiments. The method comprises: applying an input signal to the second conductor layer; measuring an impedance of the fluid sample to determine a correction algorithm; applying an input signal to the first conductor layer to generate one or more output signals from the fluid sample; correcting the one or more output signals using the correction algorithm; and determining an amount of the first analyte and an amount of the second analyte in the fluid sample, based on the corrected one or more output signals.

### BRIEF DESCRIPTION OF THE DRAWINGS

Specific embodiments of the invention will now be described in connection with the accompanying drawings, of which:

FIG. 1a is an exploded view of a multi-analyte test strip according to one embodiment of the invention;

FIG. 1b shows the assembled test strip of FIG. 1a;

FIG. 1c shows the dimensions and spacings of the elements of the test strip which are located within the sample capillary volume of the test strip of FIG. 1b;

FIG. 2 shows main effects plots from an ‘analysis of variance’ (ANOVA) of the current responses taken from working electrode currents from test strips according to FIG. 1b;

FIG. 3 shows a comparison of equivalent blood glucose measurements from different reference glucose and haematocrit levels, obtained using test strips of FIG. 1b, by means of either (i) a 5-second current based calibration algorithm with no haematocrit correction, or (ii) a Principle Components regression based algorithm which corrects for the effect of haematocrit;

FIG. 4 shows comparable Bland Altman plots of the measurement bias versus the reference glucose values for the results of FIG. 3;

FIG. 5 shows the calibrated results of a blood β-hydroxybutyrate measurement made using test strips of FIG. 1b, where the blood ketone level and the blood glucose level were separately varied, but where the blood haematocrit remained fixed, and where an end current calibration algorithm for β-hydroxybutyrate measurement was used;

FIG. 6 shows the comparison of equivalent blood β-hydroxybutyrate measurements made using test strips of FIG. 1b, where the blood ketone level and the blood glucose level were separately varied, and where the blood haematocrit was also separately varied, and where either (i) a 5-second current based calibration algorithm with no haematocrit correction, or (ii) a Principle Components regression algorithm which corrects for the effect of haematocrit, was used;

FIG. 7a is an exploded view of a multi-analyte test strip according to a further embodiment of the invention;

FIG. 7b shows the assembled test strip of FIG. 7a;

FIG. 7c shows the dimensions and spacings of the elements of the test strip which are located within the sample capillary volume of test strip of FIG. 7b;

FIG. 8 shows main effects plots from an ‘analysis of variance’ (ANOVA) of the current responses taken from working electrode currents from test strips of FIG. 7b;

FIG. 9 shows the process steps involved in first deriving and then applying a Principle Components regression algorithm of the type used to correct for the effect of haematocrit;

FIG. 10 shows a comparison of equivalent blood glucose measurements from different reference glucose and haematocrit levels, obtained using test strips of FIG. 7b, by means of either (i) a 5-second current based calibration algorithm with no haematocrit correction, or (ii) a Principle Components regression based algorithm which corrects for the effect of haematocrit;

FIG. 11 shows comparable Bland Altman plots of the measurement bias versus the reference haematocrit values for the results of FIG. 10, separated by individual reference glucose level;

FIG. 12a shows the calibrated results of a blood β-hydroxybutyrate measurement made using test strips of FIG. 7b, where the blood ketone level and the blood glucose level were separately varied, but where the blood haematocrit remained fixed, and where an end current calibration algorithm for β-hydroxybutyrate measurement was used;

FIG. 12b shows the results of FIG. 12a with variations in reference glucose highlighted, demonstrating insensitivity of the β-hydroxybutyrate measurement to the changes in the reference glucose;

FIG. 12c shows a prediction of the effect of changes in haematocrit on the measured ketone response based on the ANOVA of FIG. 8;

FIG. 13 shows the comparison of equivalent blood β-hydroxybutyrate measurements made using test strips of FIG. 7b, where the blood ketone level and the blood glucose level were separately varied, and where the blood haematocrit was also separately varied, and where either (i) an end current based calibration algorithm with no haematocrit correction, or (ii) an end current based calibration algorithm which was corrected using a haematocrit prediction derived from the Principle Components derived using the glucose measurement electrodes, was used;

FIG. 14a shows a multi-analyte test strip according to a further embodiment of the invention, and the dimensions and spacings of the elements of the test strip which are located within the sample capillary volume of test strip;

FIG. 14b shows is an exploded view of the functional layers which make up the test strip of FIG. 14a; and

FIG. 14c shows cross sections of the test strip of FIG. 14a taken along one edge and along a centre line thereof.

### DETAILED DESCRIPTION OF SPECIFIC EMBODIMENTS

The present invention seeks to provide an improved test device for measuring amounts of a plurality of analytes in a fluid sample. Whilst various embodiments of the invention are described below, the invention is not limited to these embodiments, and variations of these embodiments may well fall within the scope of the invention which is to be limited only by the appended claims.

Throughout this specification, reference is made to directional terms such as “above” and “below”, or “upper” and “lower”. References made to such terms are purely indicative of relative positions of the features of embodiments disclosed herein. For example, wherever there is mention of a cover above a spacer layer and an insulator layer below the spacer layer, it should be understood that the cover and the insulator layer are formed on opposite sides of the spacer layer. That is, directional terms such as those described herein do not refer to a direction relative to a viewpoint of an observer, but instead should be considered in all aspects as relative terms.

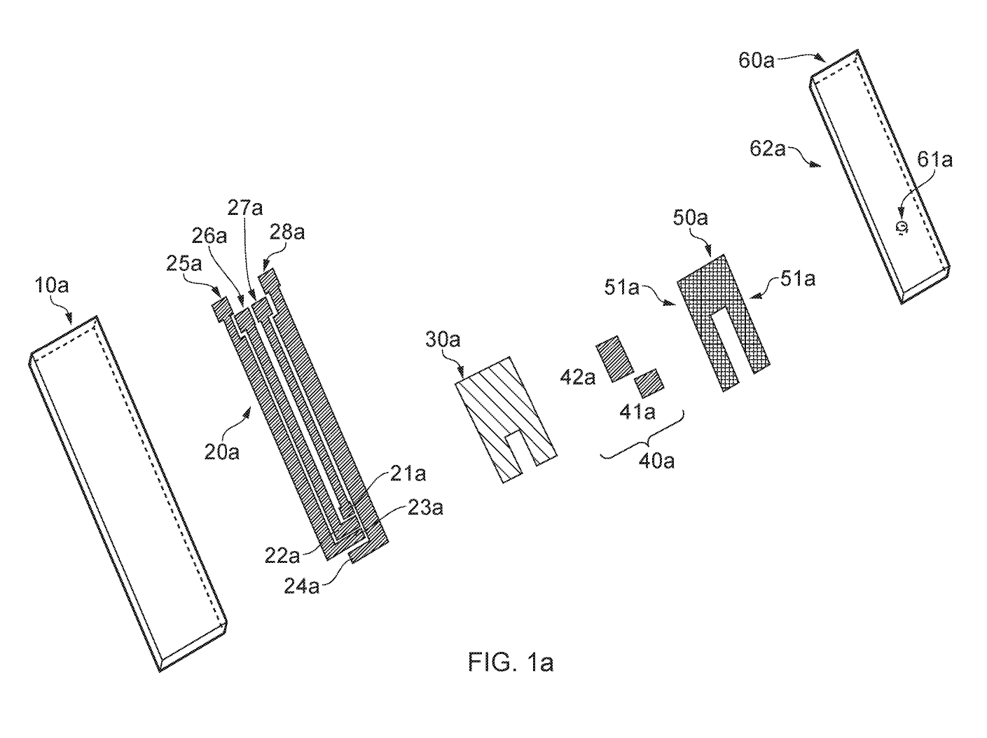

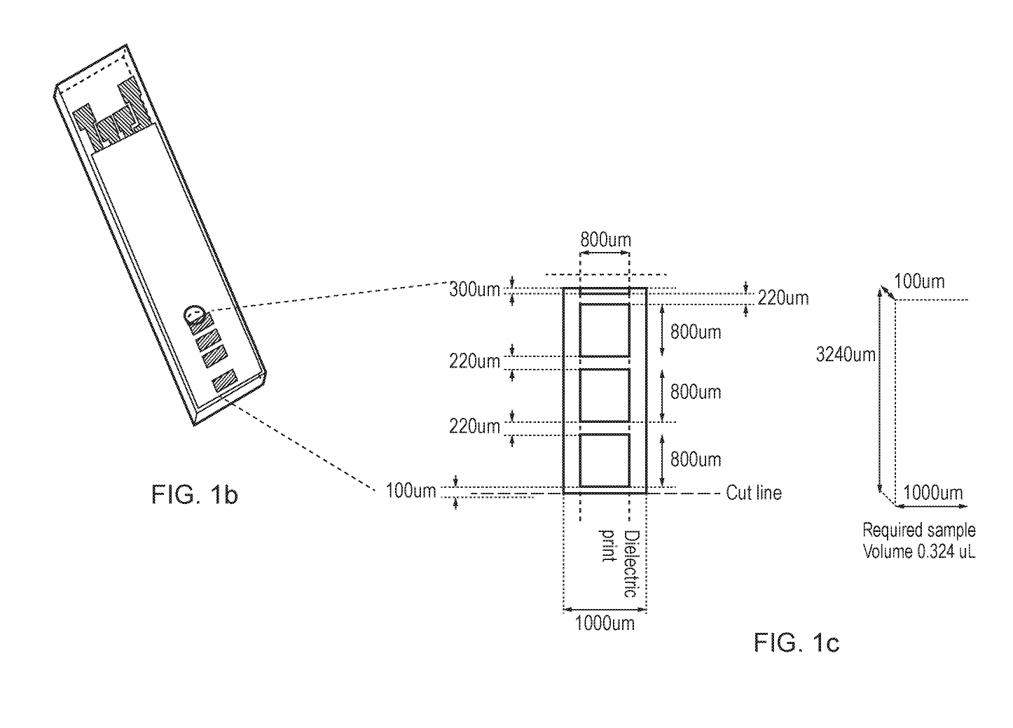

FIGS. 1a-1c show an illustrative embodiment of the present invention. In particular, FIG. 1b shows a very low volume multi-analyte test strip. FIG. 1a shows the individual elements of the very low volume multi-analyte test strip, and the layers by which this is built up. FIG. 1c shows the typical dimensions of the main elements within the measurement volume of the multi-analyte test strip.

The positioning outlined successfully achieves a required sample volume of <0.33 uL whilst optimally allowing manufacturing tolerances that are consistent with high-speed, high-volume, low-cost manufacturing processes, and an arrangement of functional components that enables accurate measurements predominantly free of crosstalk errors, while simultaneously enabling appropriate haematocrit mitigation strategies to be effected.

Referring to FIG. 1a, a base substrate layer 10a upon which is disposed a conductive electrode layer 20a is patterned to form a plurality of working electrodes (21a,22a,24a) and a reference/counter electrode (23a) for interaction with a test sample and corresponding electrode contacts (25a,26a,27a,28a) for connection to a test meter. The function of the reference/counter electrode 23a is to maintain a substantially fixed reference potential relative to the working electrodes, while functioning as a return path for currents generated by the working electrode reactions. This can be effected by means of employing further electrochemical reactions involving almost insoluble electroactive species to balance charge building up at the reference/counter electrode (RCE). Alternatively it can be effected by exploiting an excess of soluble redox active mediator species in the vicinity of the reference/counter electrode to balance charge building up at the reference/counter electrode (RCESM). A reference/counter electrode may be a reference/counter electrode with a soluble mediator (RCESM). An optional dielectric layer (30a) serves to precisely define the electrode areas exposed to the sample during measurement. Element 40a comprises a reagent layer, which is comprised of a plurality of reagent formulations (41a,42a) disposed so as to cover elements of the exposed electrodes (21a,22a,23a,24a). A capillary spacer layer 50a is coated on both sides with an adhesive (51a). The test device further comprises a cover layer 60a with a hydrophilic coating on the underside (62a) and a vent hole (61a) through which air escapes when the capillary is filled with analyte containing sample. In this embodiment, the cover layer 60a cut out of upper edge 50a almost completely masks off electrode 21a, such that only a sliver of this electrode is exposed to the sample upon filling of the test strip below the vent hole 61a.

Reagent layer 41a is designed to be a moderate to slow dissolving and moderate to slow diffusing reagent composition which is electrochemically sensitive to beta-hydroxybutyrate, which completely covers electrode 24a and which will not impinge upon electrode 23a. Reagent layer 42a is designed to be a rapidly dissolving and rapidly diffusing reagent composition which is electrochemically sensitive to beta-D-Glucose, and is designed to cover at least the bottom portion of electrode 21a, to completely cover electrode 22a and to cover at least a portion of electrode 23a whilst not impinging on any of electrode 24a. With the addition of reagent layers 41a and 42a to electrodes (21a,22a,23a,24a), conductive electrode layer 20a is arranged to provide error correction information for correcting a measurement of a response of a blood sample with at least one of reagent layers 41a and 42a, as will be described below. This arrangement uses a single glucose measurement reagent chemistry with very rapid solvation and diffusion characteristics, along with a single ketone measurement reagent with much more progressive dissolution and diffusion characteristics. GB1419799.0, filed on 6 Nov. 2014 and incorporated herein by reference in its entirety, describes how two differently solvating electrodes for the same analyte can be used to algorithmically effect a precise haematocrit compensated measurement.

The presence of a rapidly solvating glucose measurement electrode maximises the underlying measurement precision that can be obtained from the system. When this response is taken in combination with the response signal from the more progressively solvating measurement glucose electrode, the amount of ‘correction’ information that can be derived from which to correct for sample variation effects (such as haematocrit variation) is also optimised. An algorithmic combination of the two signals therefore allows highly precise haematocrit compensated measurements to be effected.

In the present embodiment, however, it will become apparent that the same basic haematocrit mitigation approach, relying on the differential solvation and diffusion properties of the two measurement electrodes, can be implemented even in the event wherein the rapidly solvating and slower solvating electrodes are actually targeted towards different analytes. The multi-functional use of this single element thereby effectively frees up sample volume from the design into which the second analyte measurement components can be accommodated, allowing for the performance of accurate, haematocrit corrected multi-analyte measurements within a sample volume that is equivalent to or lower than that normally required by a single analyte SMBG measurement system.

In this embodiment, fill assurance is accomplished by means of fluid reaching the bottom sliver portion of electrode 21a which is positioned adjacent to the vent hole 61a. The presence of an electrochemical signal thereat confirms that adequate sample to completely cover the measurement electrodes (22a, 23a, 24a) has been introduced.

Whilst this layout and arrangement is particularly preferred with regard to the minimisation of carry over crosstalk and diffusional crosstalk, other arrangements can also be effective for the multi-analyte measurement with appropriate care given to the separation and dissolution rates of the respective layers. Specifically, the location of the slow dissolution ketone reagent layer (41a) away from the RCESM (23a) and more specifically, away from the glucose electrode (22a), is particularly beneficial.

### Manufacture of the Test Strips

Test strips were manufactured according to the design described above. The following method may be used to fabricate a plurality of test strips which may be referred to as a batch of test strips or a lot of test strips. However, it may be understood that the method may also be used to fabricate just one test strip. In other words, the following method may be used to manufacture one test strip or a plurality of test strips.

Specifically, sheets of substrate layer (10a) comprised of 350 μm thickness polyester were screen printed with a layer (20a) of conductive carbon ink and then a layer (30a) of dielectric ink. Reagent layers (41a,42a) were screen printed on top using compositions comprised as follows.

Reagent layer (41a) ink (covering electrode 24a): 3 g of MBR hydroxyethyl cellulose and 6 g of 250LR hydroxyethylcellulose was dissolved in 0.1M pH7.4 Tris buffer to a volume of 100 ml. To this was added 0.5 g Tween 20, 10 g of Ruthenium (III) Hexaammine Trichloride, 0.13 g of Flavin Mononucleotide, 1.3 g of NAD, 1 g of diaphorase and 1 g of beta-hydroxybutyrate dehydrogenase. A dry film weight of about 8 gsm was targeted. In this reagent layer, the high ratio of film formers to non-film formers and the relatively high film weight ensures a slower, more progressive dissolution and solvation of the reagent layer in use.

Reagent layer (42a) ink (covering electrodes 22a,23a, impinging on the lower edge of 21a): 4 g of MBR hydroxyethyl cellulose was dissolved in 0.1M pH7.4 phosphate buffer to a volume of 100 ml. To this was added 1.5 g of fumed silica, 0.5 g of Tergitol NP9, 15 g of potassium Hexacyanoferrate(III), and 2 g of FAD-Glucose Dehydrogenase. A dry film weight of about 6 gsm was targeted. In this reagent layer, the low ratio of film formers to non-film formers and the relatively low film weight ensures a moderately rapid diffusion and solvation of the reagent layer in use.

Capillary fill test strips were then formed by first cutting the printed sheets into rows, then laminating these with a multi-layered self-adhesive laminate (50a,60a). The laminated rows were slit into individual test strips, and these were loaded into desiccated vials for storage. Dimensions were as outlined in FIG. 1c and the resulting test strips had a capillary sample volume of <0.33 μL.

The strips were tested as follows. The contact ends of the test strips (25a,26a,27a,28a) were first inserted into a test meter to make electrical connections with the electronics contained within. A potential of +300 mV was thereby simultaneously applied across all three working electrodes (21a,22a,24a) relative to the RCESM (22a). The test strips were filled with blood sample by capillary action, while current was measured simultaneously from each electrode. As soon as current exceeding 100 nA was detected from the downstream electrode (21a), timing of the measurement began (t=0 seconds) and a time based response characteristic current was measured from each of the three electrodes simultaneously. At t=85, the applied potential was reversed to −300 mV, and current measurements continued for a further 2s. The measurement steps described above were completed for multiple electrodes using blood samples that had different, known concentrations of beta-D-glucose, beta-hydroxybutyrate (ketone) concentrations, and different, known sample haematocrits.

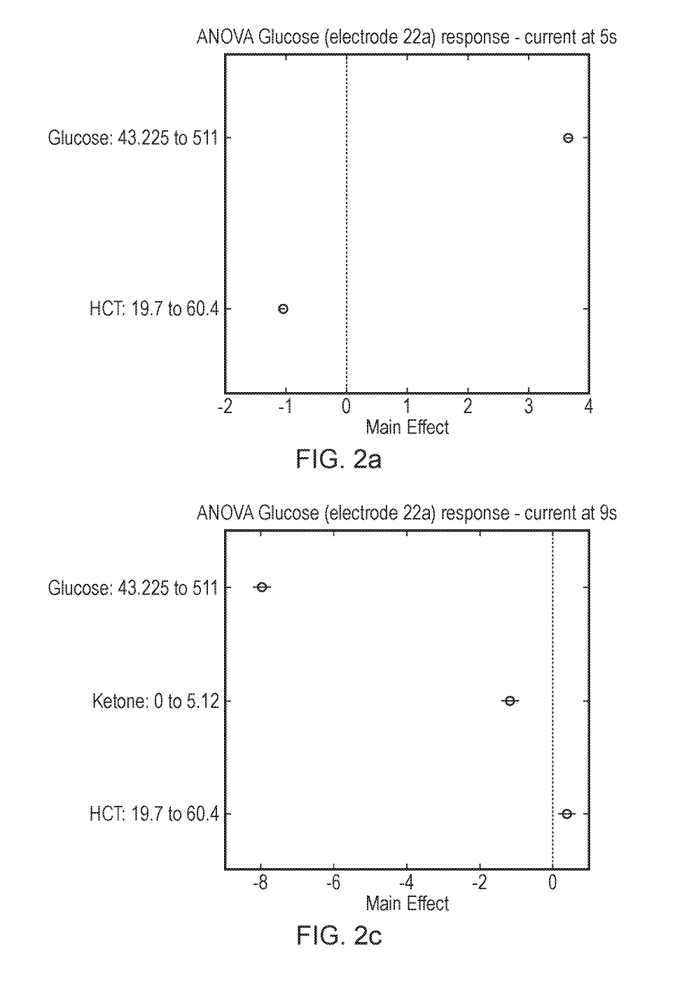

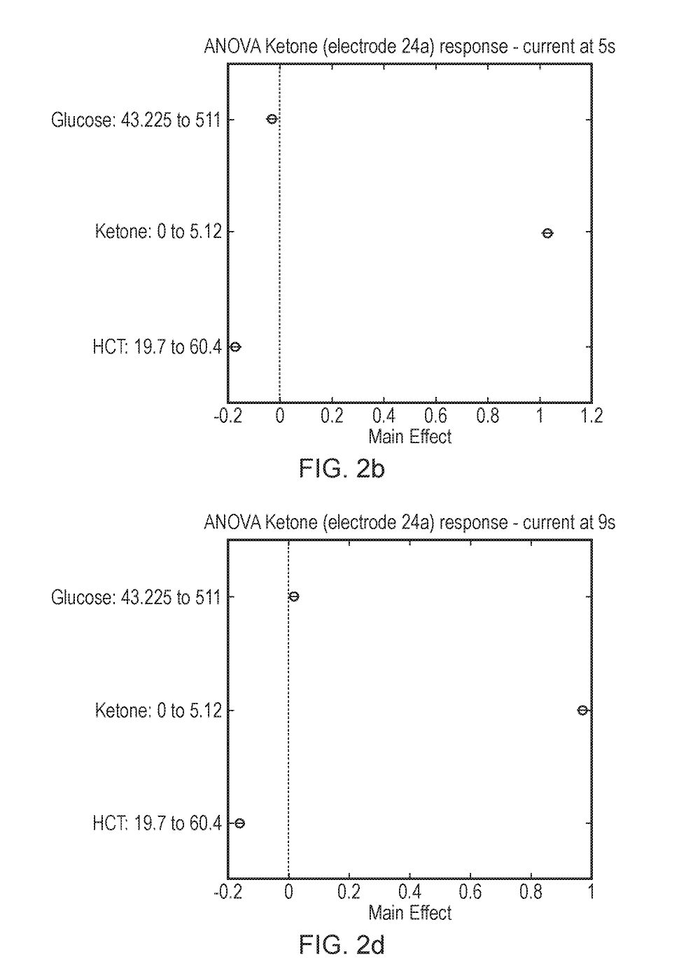

Analysis of variables (ANOVA) was performed for the current response characteristics at example sample times for each of the three electrodes, at time points before and after the potential was reversed, with the known glucose concentrations, ketone concentrations and sample haematocrit treated as dependent variables, and the resulting current treated as the response variable. The resulting main effects plots for the current responses are shown in FIGS. 2a-2d.

FIG. 2a shows the main effects plot for the glucose measurement electrode (22a) at 5 seconds during the forward pulse. It shows a response that is highly sensitive to the glucose concentration, insensitive to the concentration of ketone (therefore ostensibly free of crosstalk) and somewhat sensitive to the effect of sample haematocrit.

FIG. 2b shows the main effects plot for the ketone measurement electrode (24a) at 5 seconds during the forward pulse. It shows a response that is highly sensitive to the ketone concentration, largely insensitive to the concentration of glucose (therefore ostensibly free of crosstalk) and somewhat sensitive to the effect of sample haematocrit.

FIG. 2c shows the main effects plot for the glucose measurement electrode (22a) at 9 seconds during the reverse potential pulse. It shows a response that is highly sensitive to the glucose concentration, somewhat sensitive to the concentration of ketone (therefore suffering from electrolyte potential crosstalk) and somewhat insensitive to the effect of sample haematocrit, with a haematocrit sensitivity that is significantly different to that which was observed during the forward pulse.

FIG. 2d shows the main effects plot for the ketone measurement electrode (24a) at 9 seconds during the reverse potential pulse. It shows a response that is highly sensitive to the ketone concentration, largely insensitive to the glucose concentration (and therefore ostensibly free of crosstalk) but somewhat sensitive to the effect of sample haematocrit.

It now becomes apparent that the test strip and testing configuration outlined above can, simultaneously demonstrate the capability for (a) a substantially glucose concentration independent ketone measurement response, which is nevertheless haematocrit sensitive (FIGS. 2b and 2d) and (b) a substantially ketone concentration independent glucose measurement response which is also nevertheless haematocrit sensitive (FIGS. 2a), and (c) two simultaneously derived glucose dependent response signals characterised in that they have differentiated haematocrit sensitivities (FIGS. 2c and 2a) albeit one of which is susceptible to electrolyte potential crosstalk from the ketone electrode (11c). The combination of (a) and (c) can be used together as means by which response independent haematocrit information can be extracted, for example by first correcting the signal of FIG. 2c for variations in ketone level (derived from the signals of FIGS. 2b and 2d), and then using this modified signal with the unmodified signal of FIG. 2c for the purpose of correcting the haematocrit effect for either or both of the substantially glucose independent ketone measurement signal of (a), and/or the substantially ketone independent glucose signals of (b).

This shows that the design configuration as employed has adequately mitigated ‘carry over’ and ‘diffusional’ crosstalk effects from the design, despite the very small sample volume achieved, and in a manner which is compatible with high volume, low cost manufacturing methods.

It also becomes apparent in light of this ANOVA that, while potential ‘pulsing’ or potential reversal does lead to the introduction of confounding measurement signals from across the different analyte electrodes within a multi-analyte system (i.e. electrolyte potential crosstalk effects), there exist some means by which this detrimental effect can be advantageously offset to a useful extent. This may be achieved with appropriate designs (and notably where these designs are not separately susceptible to ‘carry over’ and ‘diffusional’ crosstalk), by first using signals from the synchronised forward pulse of applied potential and then intelligently combining these with the reverse pulse signals, such that useful haematocrit correction information can still be extracted, through the implementation of signal processing methodologies and algorithms that are specifically designed to take account of the electrolyte potential crosstalk effects that were thereby introduced.

### Example 1

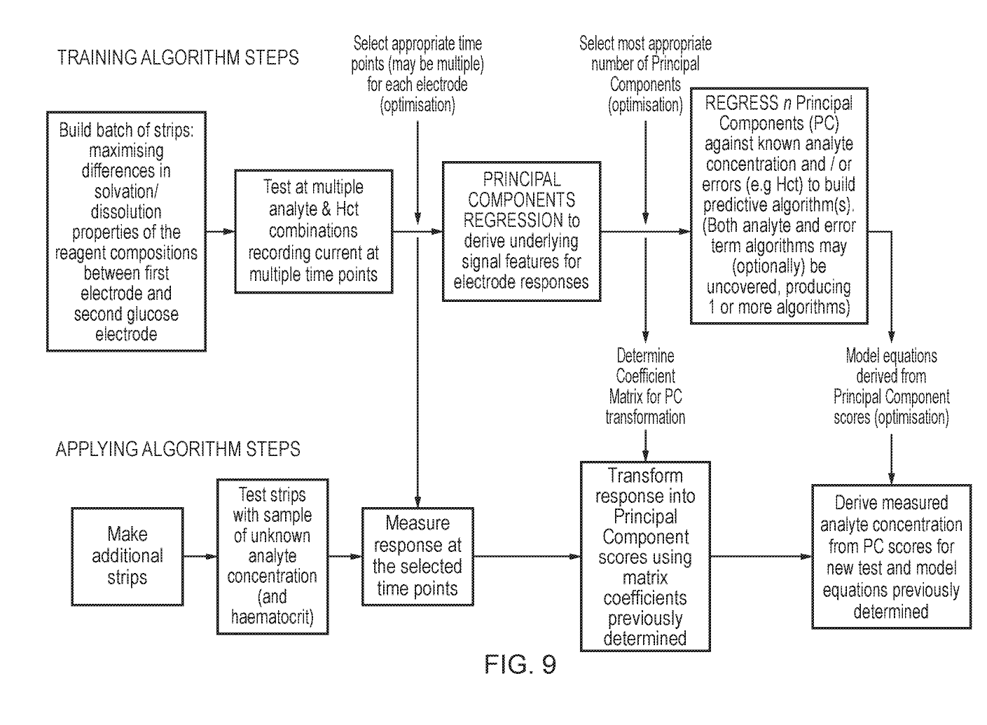

Test strips made according to the above embodiment were then tested for 10 seconds with +300 mV applied between all of the working electrodes (21a,22a,24a) and the reference/counter electrode (23a) for the first 8 seconds and −300 mV applied between all of the working electrodes (21a,22a,24a) and the reference/counter electrodes (23a) for the final two seconds. A haematocrit corrected glucose response was derived from a ‘Principle Components’ (PC) Calibration scheme derived according to the procedure outlined in FIG. 9, which in this case exploited the intentionally designed ‘differential’ solvation and diffusional properties of the glucose ink (rapid solvation—electrode 22a) and the ketone ink (progressive solvation—electrode 24a) at times before and after the applied potential was reversed, to derive a haematocrit corrected glucose response which simultaneously maximised the measurement precision and minimised/eliminated any residual crosstalk effects.

In this embodiment, a two-stage algorithm was used. The first algorithm (Algorithm 1) was trained using all reference glucose levels. The second algorithm (Algorithm 2) was trained only with reference glucose values below 200 mg/dL. When applying the algorithm to produce a calibrated response, Algorithm 1 was first used, and only where Algorithm 1 indicated a glucose result of 130 mg/dL or below was a result derived from Algorithm 2 substituted.

For Algorithm 1, the 23 raw signal inputs were studentized using the following means and standard deviations to derive the algorithm inputs:

| | elec- | | | |
|---|---|---|---|---|
| Algorithm_Input | trode | time_sec | mean_Amps | st_dev_Amps |
| 1 | 24a | 1 | 5.663E−07 | 2.833E−07 |
| 2 | 24a | 2 | 5.543E−07 | 3.272E−07 |
| 3 | 24a | 3 | 5.297E−07 | 3.427E−07 |
| 4 | 24a | 4 | 5.073E−07 | 3.459E−07 |
| 5 | 24a | 5 | 4.877E−07 | 3.429E−07 |
| 6 | 24a | 6 | 4.705E−07 | 3.367E−07 |
| 7 | 24a | 8.5 | 2.457E−07 | 3.208E−07 |
| 8 | 24a | 9 | 2.698E−07 | 3.239E−07 |
| 9 | 24a | 9.2 | 2.731E−07 | 3.239E−07 |
| 10 | 24a | 9.2 | 2.731E−07 | 3.239E−07 |
| 11 | 24a | 9.4 | 2.751E−07 | 3.235E−07 |
| 12 | 24a | 9.6 | 2.762E−07 | 3.228E−07 |
| 13 | 24a | 9.8 | 2.767E−07 | 3.220E−07 |
| 14 | 22a | 3 | 2.736E−06 | 1.581E−06 |
| 15 | 22a | 4 | 2.450E−06 | 1.418E−06 |
| 16 | 22a | 5 | 2.265E−06 | 1.310E−06 |
| 17 | 22a | 5.3 | 2.221E−06 | 1.285E−06 |
| 18 | 22a | 5.6 | 2.181E−06 | 1.261E−06 |
| 19 | 22a | 5.9 | 2.144E−06 | 1.240E−06 |
| 20 | 22a | 6.2 | 2.110E−06 | 1.221E−06 |
| 21 | 22a | 6.8 | 2.050E−06 | 1.187E−06 |
| 22 | 22a | 8.9 | −6.083E−06 | 2.950E−06 |
| 23 | 22a | 9.5 | −4.194E−06 | 2.222E−06 |

The coefficient matrix from which the PC model inputs (x1:x23) were derived by matrix multiplication of the 23 algorithm inputs for Algorithm 1 was as follows:

| INPUT | 1 | 2 | 3 | 4 | 5 | 6 | 7 | 8 | 9 | 10 | 11 | 12 |
|---|---|---|---|---|---|---|---|---|---|---|---|---|
| 1 | 0.2561 | −0.0941 | −0.1904 | 0.7040 | 0.4553 | −0.0924 | −0.1826 | 0.3271 | 0.1065 | −0.1576 | −0.0078 | 0.0153 |
| 2 | 0.2680 | −0.0712 | −0.0845 | 0.3784 | −0.0908 | 0.0679 | 0.1282 | −0.4907 | −0.1806 | 0.6062 | −0.0029 | −0.1585 |
| 3 | 0.2707 | −0.0647 | −0.0232 | 0.2063 | −0.2931 | 0.1402 | 0.1549 | −0.3537 | −0.0226 | −0.2659 | 0.0039 | 0.2229 |
| 4 | 0.2714 | −0.0628 | 0.0099 | 0.0918 | −0.3661 | 0.1571 | 0.0799 | −0.0416 | 0.0902 | −0.4742 | 0.0211 | 0.1287 |
| 5 | 0.2715 | −0.0628 | 0.0289 | 0.0066 | −0.3743 | 0.1443 | −0.0316 | 0.2751 | 0.0666 | −0.1221 | −0.0021 | −0.2800 |
| 6 | 0.2712 | −0.0638 | 0.0413 | −0.0595 | −0.3586 | 0.1127 | −0.1695 | 0.5731 | −0.0598 | 0.4743 | −0.0117 | 0.0967 |
| 7 | 0.2727 | −0.0526 | 0.0782 | −0.1868 | 0.1111 | −0.1232 | 0.0015 | −0.1361 | 0.8098 | 0.2213 | −0.0312 | 0.3296 |
| 8 | 0.2733 | −0.0493 | 0.0465 | −0.1847 | 0.1390 | −0.0417 | 0.0030 | −0.0726 | 0.1229 | −0.0657 | −0.0429 | −0.4386 |
| 9 | 0.2734 | −0.0489 | 0.0403 | −0.1846 | 0.1511 | −0.0507 | 0.0038 | −0.0424 | −0.0453 | −0.0662 | −0.0292 | −0.3014 |
| 10 | 0.2734 | −0.0489 | 0.0403 | −0.1846 | 0.1511 | −0.0507 | 0.0038 | −0.0424 | −0.0453 | −0.0662 | −0.0292 | −0.3014 |
| 11 | 0.2734 | −0.0488 | 0.0361 | −0.1838 | 0.1604 | −0.0651 | 0.0046 | −0.0149 | −0.1789 | −0.0580 | −0.0012 | −0.0693 |
| 12 | 0.2734 | −0.0488 | 0.0332 | −0.1827 | 0.1698 | −0.0818 | 0.0022 | 0.0030 | −0.2948 | −0.0311 | 0.0423 | 0.2464 |
| 13 | 0.2734 | −0.0489 | 0.0310 | −0.1812 | 0.1760 | −0.0985 | −0.0024 | 0.0221 | −0.3689 | 0.0040 | 0.0924 | 0.5097 |
| 14 | 0.0645 | 0.3133 | 0.1129 | 0.0789 | 0.0627 | −0.0759 | 0.6767 | 0.2186 | 0.0552 | 0.0557 | 0.5112 | −0.0685 |
| 15 | 0.0643 | 0.3132 | 0.1345 | 0.0643 | 0.0319 | −0.0416 | 0.3440 | 0.1266 | −0.0124 | −0.0096 | −0.2939 | 0.0533 |
| 16 | 0.0640 | 0.3133 | 0.1442 | 0.0517 | 0.0099 | −0.0028 | 0.0431 | 0.0136 | −0.0347 | −0.0066 | −0.3567 | 0.0221 |
| 17 | 0.0639 | 0.3133 | 0.1457 | 0.0487 | 0.0052 | 0.0039 | −0.0397 | −0.0116 | −0.0201 | −0.0082 | −0.3380 | 0.0031 |
| 18 | 0.0639 | 0.3133 | 0.1476 | 0.0450 | 0.0006 | 0.0156 | −0.1222 | −0.0401 | −0.0236 | 0.0056 | −0.2154 | 0.0260 |
| 19 | 0.0637 | 0.3133 | 0.1489 | 0.0415 | −0.0043 | 0.0276 | −0.2029 | −0.0618 | −0.0159 | 0.0014 | −0.0147 | 0.0077 |
| 20 | 0.0637 | 0.3133 | 0.1498 | 0.0382 | −0.0105 | 0.0365 | −0.2800 | −0.0829 | −0.0051 | −0.0060 | 0.1482 | −0.0151 |
| 21 | 0.0633 | 0.3133 | 0.1514 | 0.0319 | −0.0201 | 0.0587 | −0.4114 | −0.1261 | 0.0303 | −0.0280 | 0.5714 | −0.0335 |
| 22 | −0.0760 | −0.2905 | 0.6284 | 0.2073 | −0.2076 | −0.6489 | −0.0426 | −0.0183 | −0.0526 | −0.0294 | 0.0127 | −0.0402 |
| 23 | −0.0847 | −0.2884 | 0.6176 | 0.0693 | 0.2903 | 0.6572 | 0.0683 | 0.0238 | 0.0079 | 0.0284 | 0.0017 | 0.0315 |
| | INPUT | 13 | 14 | 15 | 16 | 17 | 18 | 19 | 20 | 21 | 22 | 23 |
| | 1 | 0.0303 | −0.0127 | −0.0016 | 0.0047 | −0.0019 | 0.0001 | 0.0026 | −0.0008 | −0.0025 | 0.0020 | 0.0000 |
| | 2 | −0.2557 | 0.1032 | −0.0250 | −0.0227 | 0.0094 | 0.0033 | −0.0106 | 0.0052 | 0.0031 | −0.0061 | 0.0000 |
| | 3 | 0.6179 | −0.2932 | 0.1812 | 0.0498 | −0.0029 | −0.0064 | 0.0254 | −0.0107 | 0.0020 | 0.0065 | 0.0000 |
| | 4 | −0.3025 | 0.4862 | −0.3990 | −0.0547 | −0.0707 | 0.0109 | −0.0344 | 0.0238 | 0.0019 | −0.0034 | 0.0000 |
| | 5 | −0.4546 | −0.4573 | 0.3917 | 0.0167 | 0.1216 | −0.0252 | 0.0126 | −0.0313 | −0.0050 | 0.0015 | 0.0000 |
| | 6 | 0.3584 | 0.1652 | −0.1454 | 0.0078 | −0.0567 | 0.0179 | 0.0027 | 0.0131 | 0.0015 | −0.0006 | 0.0000 |
| | 7 | −0.0950 | −0.0607 | 0.0526 | 0.0096 | −0.0622 | −0.0171 | −0.0374 | 0.0090 | 0.0115 | 0.0047 | 0.0000 |
| | 8 | 0.1981 | 0.1309 | −0.1265 | −0.0435 | 0.5422 | 0.2096 | 0.4568 | 0.1521 | −0.0368 | −0.0370 | 0.0000 |
| | 9 | 0.1047 | 0.0503 | −0.0225 | 0.0164 | −0.2024 | −0.1700 | −0.2269 | −0.3512 | −0.0525 | 0.0121 | 0.7071 |
| | 10 | 0.1047 | 0.0503 | −0.0225 | 0.0164 | −0.2024 | −0.1700 | −0.2269 | −0.3512 | −0.0525 | 0.0121 | −0.7071 |
| | 11 | 0.0028 | −0.0434 | 0.0375 | 0.0060 | −0.2025 | −0.1124 | −0.2733 | 0.8149 | 0.1409 | 0.0427 | 0.0000 |
| | 12 | −0.1488 | −0.0872 | 0.0772 | −0.0493 | −0.3946 | 0.5957 | 0.3621 | −0.1360 | 0.0650 | −0.0027 | 0.0000 |
| | 13 | −0.1602 | −0.0315 | 0.0024 | 0.0431 | 0.5229 | −0.3363 | −0.0528 | −0.1370 | −0.0765 | −0.0318 | 0.0000 |
| | 14 | 0.0130 | −0.1669 | −0.2314 | 0.0926 | −0.0185 | 0.0012 | 0.0071 | −0.0026 | 0.0217 | 0.0109 | 0.0000 |
| | 15 | 0.0467 | 0.4009 | 0.5411 | −0.4289 | 0.0569 | 0.0271 | −0.0687 | −0.0064 | −0.0061 | −0.0210 | 0.0000 |
| | 16 | −0.0571 | 0.0844 | 0.0527 | 0.6175 | −0.1791 | −0.2406 | 0.3559 | 0.0781 | −0.3121 | 0.1689 | 0.0000 |
| | 17 | −0.0134 | −0.0907 | −0.1531 | 0.2880 | 0.1396 | 0.1762 | −0.2134 | −0.0950 | 0.5872 | −0.4393 | 0.0000 |
| | 18 | −0.0103 | −0.2286 | −0.2714 | −0.1476 | 0.1823 | 0.3451 | −0.3545 | −0.0157 | −0.2306 | 0.5747 | 0.0000 |
| | 19 | −0.0115 | −0.2298 | −0.2139 | −0.3743 | −0.1399 | −0.1214 | 0.0882 | 0.0883 | −0.4590 | −0.5729 | 0.0000 |
| | 20 | −0.0011 | −0.0559 | −0.0581 | −0.3123 | −0.1160 | −0.3897 | 0.3651 | −0.0597 | 0.4970 | 0.3342 | 0.0000 |
| | 21 | 0.0338 | 0.2861 | 0.3320 | 0.2657 | 0.0746 | 0.2023 | −0.1804 | 0.0129 | −0.0973 | −0.0555 | 0.0000 |
| | 22 | 0.0100 | 0.0049 | −0.0051 | 0.0001 | −0.0006 | 0.0013 | 0.0007 | −0.0001 | −0.0019 | 0.0002 | 0.0000 |
| | 23 | −0.0099 | −0.0048 | 0.0026 | 0.0003 | 0.0006 | −0.0011 | −0.0015 | −0.0001 | 0.0029 | −0.0002 | 0.0000 |

The regression equation coefficients, by which the measured result was derived from the 23 model inputs (x1:x23) for Algorithm 1 were as follows:

| model_term | Coefficient |
|---|---|
| (Intercept) | 2.271E+02 |
| x1 | 9.367E+00 |
| x2 | 4.945E+01 |
| x3 | −4.418E−01 |
| x4 | −9.418E+01 |
| x5 | 5.068E+01 |
| x6 | 1.151E+02 |
| x7 | −2.133E+02 |
| x8 | −2.043E+02 |
| x9 | −3.023E+02 |
| x10 | −4.286E+02 |
| x11 | −1.877E+02 |
| x12 | −1.949E+02 |
| x13 | 5.277E+02 |
| x14 | 3.747E+02 |
| x15 | 1.185E+03 |
| x16 | 1.419E+02 |
| x17 | 6.508E+02 |
| x18 | −4.374E+02 |
| x19 | 6.734E+02 |
| x20 | 6.851E+02 |
| x21 | 4.374E+02 |
| x1:x2 | 5.198E−01 |
| x1:x4 | 9.335E+00 |
| x1:x5 | 1.352E+01 |
| x1:x6 | −9.523E+00 |
| x1:x7 | 2.787E+01 |
| x1:x8 | −5.271E+01 |
| x1:x9 | 8.823E+01 |
| x1:x11 | −2.433E+02 |
| x1:x21 | −7.160E+02 |
| x2:x3 | −9.487E+00 |
| x2:x4 | −1.717E+01 |
| x2:x7 | −3.959E+01 |
| x2:x8 | −7.544E+01 |
| x2:x13 | 3.431E+02 |
| x2:x17 | 4.139E+02 |
| x3:x5 | −5.716E+01 |
| x3:x10 | −4.306E+02 |
| x3:x12 | 1.601E+03 |
| x4:x7 | 5.993E+02 |
| x4:x8 | −3.892E+02 |
| x4:x11 | 9.868E+02 |
| x4:x12 | −1.416E+03 |
| x5:x6 | −3.158E+02 |
| x5:x7 | 8.743E+02 |
| x5:x8 | 9.120E+02 |
| x5:x12 | 4.214E+03 |
| x5:x16 | −7.146E+03 |
| x5:x19 | −1.407E+04 |
| x6:x7 | −1.522E+03 |
| x6:x8 | 2.147E+03 |
| x6:x11 | −6.733E+03 |
| x6:x12 | 5.510E+03 |
| x6:x13 | −5.639E+03 |
| x7:x10 | 3.195E+03 |
| x7:x12 | 8.994E+03 |
| x7:x17 | −3.760E+04 |
| x7:x18 | −2.294E+04 |
| x8:x9 | 4.671E+03 |
| x9:x15 | 3.179E+04 |
| x9:x19 | −6.530E+04 |
| x10:x11 | 3.593E+04 |
| x12:x15 | −9.237E+04 |
| x13:x18 | −1.531E+05 |
| x14:x15 | 1.285E+05 |
| x14:x17 | 2.521E+05 |
| x14:x21 | 5.521E+05 |
| x16:x17 | −3.044E+05 |
| x17:x20 | −3.436E+05 |
| x18:x19 | −4.437E+05 |
| x20:x21 | −5.023E+05 |

For Algorithm 2, the 23 raw signal inputs used the following mean and standard deviations:

| | elec- | | | |
|---|---|---|---|---|
| Algorithm_Input | trode | time_sec | mean_Amps | st_dev_Amps |
| 1 | 24a | 1 | 6.260E−07 | 2.876E−07 |
| 2 | 24a | 2 | 5.942E−07 | 3.302E−07 |
| 3 | 24a | 3 | 5.619E−07 | 3.426E−07 |
| 4 | 24a | 4 | 5.362E−07 | 3.437E−07 |
| 5 | 24a | 5 | 5.154E−07 | 3.395E−07 |
| 6 | 24a | 6 | 4.982E−07 | 3.329E−07 |
| 7 | 24a | 8.5 | 2.602E−07 | 3.254E−07 |
| 8 | 24a | 9 | 2.807E−07 | 3.289E−07 |
| 9 | 24a | 9.2 | 2.838E−07 | 3.291E−07 |
| 10 | 24a | 9.2 | 2.838E−07 | 3.291E−07 |
| 11 | 24a | 9.4 | 2.859E−07 | 3.287E−07 |
| 12 | 24a | 9.6 | 2.873E−07 | 3.282E−07 |
| 13 | 24a | 9.8 | 2.880E−07 | 3.274E−07 |
| 14 | 22a | 3 | 1.284E−06 | 3.230E−07 |
| 15 | 22a | 4 | 1.141E−06 | 2.858E−07 |
| 16 | 22a | 5 | 1.049E−06 | 2.650E−07 |
| 17 | 22a | 5.3 | 1.027E−06 | 2.606E−07 |
| 18 | 22a | 5.6 | 1.007E−06 | 2.574E−07 |
| 19 | 22a | 5.9 | 9.882E−07 | 2.542E−07 |
| 20 | 22a | 6.2 | 9.707E−07 | 2.513E−07 |
| 21 | 22a | 6.8 | 9.390E−07 | 2.469E−07 |
| 22 | 22a | 8.9 | −3.338E−06 | 9.392E−07 |
| 23 | 22a | 9.5 | −2.242E−06 | 6.182E−07 |

The coefficient matrix from which the model inputs (x1:x23) were derived by matrix multiplication of the 23 algorithm inputs for Algorithm 2 was as follows:

| INPUT | 1 | 2 | 3 | 4 | 5 | 6 | 7 | 8 | 9 | 10 | 11 | 12 |
|---|---|---|---|---|---|---|---|---|---|---|---|---|
| 1 | 0.2494 | −0.0899 | −0.1066 | 0.7234 | 0.0235 | 0.2164 | 0.4184 | 0.3597 | −0.0144 | 0.0933 | 0.1035 | −0.1348 |
| 2 | 0.2533 | −0.1005 | −0.0178 | 0.3605 | −0.1408 | 0.0095 | −0.0485 | −0.4753 | −0.0396 | −0.2765 | −0.1783 | 0.5202 |
| 3 | 0.2535 | −0.1037 | 0.0238 | 0.1917 | −0.1903 | −0.1029 | −0.2302 | −0.4359 | 0.0238 | 0.1039 | −0.0099 | −0.1456 |
| 4 | 0.2535 | −0.1046 | 0.0477 | 0.0813 | −0.2048 | −0.1595 | −0.2981 | −0.1076 | 0.0317 | 0.1991 | 0.1023 | −0.4416 |
| 5 | 0.2531 | −0.1058 | 0.0611 | −0.0044 | −0.2191 | −0.1694 | −0.3081 | 0.2539 | 0.0572 | 0.0447 | 0.1006 | −0.1768 |
| 6 | 0.2526 | −0.1066 | 0.0699 | −0.0707 | −0.2331 | −0.1665 | −0.2861 | 0.5968 | −0.0105 | −0.1813 | −0.0578 | 0.4309 |
| 7 | 0.2529 | −0.1027 | 0.0971 | −0.1862 | 0.1642 | 0.0147 | 0.1338 | −0.1080 | 0.0982 | 0.1885 | 0.7065 | 0.3625 |
| 8 | 0.2533 | −0.1032 | 0.0768 | −0.1623 | 0.1370 | 0.0642 | 0.0974 | −0.0570 | 0.0020 | 0.0809 | 0.1108 | −0.0369 |
| 9 | 0.2533 | −0.1036 | 0.0739 | −0.1584 | 0.1379 | 0.0685 | 0.0964 | −0.0244 | −0.0152 | 0.0220 | −0.0339 | −0.0686 |
| 10 | 0.2533 | −0.1036 | 0.0739 | −0.1584 | 0.1379 | 0.0685 | 0.0964 | −0.0244 | −0.0152 | 0.0220 | −0.0339 | −0.0686 |
| 11 | 0.2533 | −0.1037 | 0.0723 | −0.1567 | 0.1389 | 0.0734 | 0.1021 | −0.0032 | −0.0259 | −0.0458 | −0.1744 | −0.0961 |
| 12 | 0.2533 | −0.1037 | 0.0711 | −0.1530 | 0.1428 | 0.0758 | 0.1016 | 0.0135 | −0.0471 | −0.1026 | −0.2813 | −0.0860 |
| 13 | 0.2533 | −0.1037 | 0.0696 | −0.1517 | 0.1446 | 0.0751 | 0.1050 | 0.0286 | −0.0467 | −0.1483 | −0.3558 | −0.0594 |
| 14 | 0.1130 | 0.3193 | 0.0156 | 0.2288 | 0.6211 | −0.2825 | −0.2571 | 0.0287 | 0.4509 | −0.2857 | 0.0453 | −0.0796 |
| 15 | 0.1119 | 0.3214 | 0.0677 | 0.1008 | 0.2526 | −0.1478 | −0.1334 | 0.0354 | −0.2733 | 0.6896 | −0.2930 | 0.2797 |
| 16 | 0.1115 | 0.3213 | 0.0985 | 0.0184 | 0.0096 | −0.0267 | −0.0260 | 0.0090 | −0.4975 | −0.0979 | 0.1149 | −0.0931 |
| 17 | 0.1110 | 0.3214 | 0.1053 | −0.0027 | −0.0455 | 0.0201 | 0.0049 | −0.0062 | −0.3397 | −0.1730 | 0.2028 | −0.1280 |
| 18 | 0.1109 | 0.3212 | 0.1128 | −0.0237 | −0.1084 | 0.0568 | 0.0476 | −0.0308 | −0.1382 | −0.2339 | 0.0873 | −0.0466 |
| 19 | 0.1107 | 0.3211 | 0.1174 | −0.0412 | −0.1656 | 0.1008 | 0.0704 | −0.0279 | 0.0708 | −0.1779 | 0.0617 | 0.0420 |
| 20 | 0.1107 | 0.3207 | 0.1225 | −0.0584 | −0.2202 | 0.1251 | 0.1067 | −0.0026 | 0.2193 | 0.0099 | −0.1133 | −0.0153 |
| 21 | 0.1103 | 0.3203 | 0.1310 | −0.0818 | −0.2970 | 0.1936 | 0.1366 | 0.0036 | 0.5143 | 0.2615 | −0.1179 | 0.0404 |
| 22 | −0.1774 | −0.1380 | 0.6992 | 0.1140 | −0.0695 | −0.5475 | 0.3730 | −0.0186 | 0.0165 | −0.0172 | −0.0475 | −0.0157 |
| 23 | −0.1946 | −0.1423 | 0.6019 | 0.1675 | 0.1496 | 0.6009 | −0.4064 | 0.0340 | −0.0137 | 0.0094 | 0.0304 | 0.0131 |
| | INPUT | 13 | 14 | 15 | 16 | 17 | 18 | 19 | 20 | 21 | 22 | 23 |
| | 1 | 0.0403 | 0.0036 | 0.0092 | 0.0513 | −0.0025 | 0.0262 | −0.0201 | 0.0041 | 0.0005 | −0.0014 | 0.0000 |
| | 2 | −0.1166 | −0.0292 | −0.0856 | −0.3276 | −0.0208 | −0.1256 | 0.1447 | −0.0253 | 0.0001 | −0.0027 | 0.0000 |
| | 3 | 0.0326 | 0.0656 | 0.2261 | 0.5817 | 0.0464 | 0.0659 | −0.4041 | 0.0193 | 0.0004 | 0.0123 | 0.0000 |
| | 4 | 0.0810 | −0.0101 | −0.1194 | −0.1714 | 0.0717 | 0.2634 | 0.6071 | 0.0912 | 0.0114 | 0.0129 | 0.0000 |
| | 5 | 0.0103 | −0.0965 | −0.1913 | −0.4318 | −0.1942 | −0.2751 | −0.5082 | −0.1767 | −0.0503 | −0.0213 | 0.0000 |
| | 6 | −0.0400 | 0.0755 | 0.1606 | 0.2943 | 0.0974 | 0.0620 | 0.1751 | 0.0878 | 0.0392 | 0.0056 | 0.0000 |
| | 7 | 0.0295 | −0.0123 | −0.0600 | −0.0620 | −0.0282 | 0.3359 | −0.0996 | 0.0019 | −0.0717 | 0.1087 | 0.0000 |
| | 8 | −0.0085 | 0.0075 | 0.1897 | 0.0028 | −0.0340 | −0.3332 | 0.1072 | 0.0017 | 0.6460 | −0.5099 | 0.0000 |
| | 9 | −0.0176 | 0.0074 | 0.1017 | 0.0689 | 0.0158 | −0.3247 | 0.1378 | 0.0075 | −0.4619 | −0.0302 | −0.7071 |
| | 10 | −0.0176 | 0.0074 | 0.1017 | 0.0689 | 0.0158 | −0.3247 | 0.1378 | 0.0075 | −0.4619 | −0.0302 | 0.7071 |
| | 11 | 0.0113 | 0.0166 | 0.0194 | 0.0325 | −0.0065 | −0.0807 | 0.0574 | −0.3279 | 0.3548 | 0.7568 | 0.0000 |
| | 12 | −0.0131 | −0.0373 | −0.1819 | −0.1011 | −0.0588 | 0.1842 | −0.2245 | 0.7841 | 0.0845 | 0.0781 | 0.0000 |
| | 13 | 0.0100 | 0.0020 | −0.1694 | −0.0060 | 0.0986 | 0.5253 | −0.1105 | −0.4755 | −0.0909 | −0.3788 | 0.0000 |
| | 14 | −0.0678 | −0.0099 | 0.0373 | 0.0204 | −0.0016 | −0.0031 | 0.0170 | 0.0082 | 0.0040 | −0.0021 | 0.0000 |
| | 15 | 0.1906 | 0.0642 | −0.0736 | −0.0459 | 0.0270 | −0.0349 | −0.0207 | −0.0085 | 0.0010 | 0.0010 | 0.0000 |
| | 16 | −0.4427 | −0.4728 | 0.1282 | 0.0958 | −0.3634 | 0.1100 | 0.0579 | −0.0298 | −0.0126 | 0.0039 | 0.0000 |
| | 17 | −0.2081 | 0.3178 | −0.0508 | −0.1339 | 0.6917 | −0.0777 | −0.1284 | 0.0291 | 0.0253 | 0.0217 | 0.0000 |
| | 18 | 0.2829 | 0.6119 | −0.1444 | 0.0826 | −0.5300 | −0.0019 | 0.0691 | −0.0158 | −0.0184 | −0.0313 | 0.0000 |
| | 19 | 0.5509 | −0.5165 | −0.3307 | 0.2102 | 0.1892 | −0.1467 | 0.0339 | −0.0006 | 0.0319 | −0.0199 | 0.0000 |
| | 20 | 0.2085 | −0.0593 | 0.7135 | −0.3569 | 0.0178 | 0.1907 | −0.0674 | 0.0301 | −0.0529 | 0.0356 | 0.0000 |
| | 21 | −0.5159 | 0.0644 | −0.2777 | 0.1288 | −0.0303 | −0.0393 | 0.0388 | −0.0129 | 0.0215 | −0.0091 | 0.0000 |
| | 22 | −0.0084 | −0.0011 | −0.0052 | 0.0044 | 0.0068 | −0.0189 | 0.0027 | 0.0005 | 0.0023 | −0.0037 | 0.0000 |
| | 23 | 0.0076 | 0.0005 | 0.0076 | −0.0035 | −0.0058 | 0.0137 | −0.0017 | −0.0011 | −0.0018 | 0.0025 | 0.0000 |

The regression equation coefficients, by which the measured result was derived from the 23 model inputs (x1:x23) for Algorithm 2 were as follows:

| model_term | Coefficient |
|---|---|
| (Intercept) | 73.895 |
| x1 | 2.765 |
| x2 | 6.625 |
| x3 | 2.527 |
| x4 | −18.196 |
| x5 | −40.501 |
| x6 | 48.576 |
| x7 | 4.006 |
| x8 | −42.553 |
| x9 | 27.791 |
| x11 | −130.864 |
| x12 | −146.943 |
| x13 | −28.207 |
| x14 | −95.854 |
| x15 | −19.001 |
| x16 | 105.413 |
| x17 | −61.581 |
| x18 | −214.259 |
| x19 | −200.154 |
| x20 | −237.899 |
| x21 | 186.869 |
| x22 | −41.994 |
| x1:x4 | 2.174 |
| x1:x15 | −36.318 |
| x2:x3 | −0.841 |
| x2:x15 | −61.495 |
| x2:x18 | 65.041 |
| x2:x22 | 190.492 |
| x3:x5 | −10.478 |
| x3:x7 | −15.060 |
| x3:x9 | −48.511 |
| x3:x11 | −144.809 |
| x3:x12 | −183.177 |
| x3:x18 | −192.781 |
| x4:x5 | 53.242 |
| x4:x12 | 324.161 |
| x5:x7 | −79.614 |
| x5:x9 | 195.658 |
| x6:x9 | −187.630 |
| x6:x14 | 1223.015 |
| x7:x8 | −532.589 |
| x7:x12 | −924.137 |
| x7:x18 | −2181.213 |
| x8:x18 | −6094.143 |
| x8:x21 | −11113.626 |
| x9:x12 | −3916.767 |
| x9:x21 | 14499.637 |
| x11:x21 | 22225.365 |
| x12:x15 | 20502.242 |
| x13:x20 | 23164.963 |
| x16:x17 | −44683.805 |
| x16:x20 | −49516.358 |
| x18:x21 | 61291.193 |
| x19:x20 | −89886.015 |

The algorithms described above were used to produce calibrated measurements from tests carried out using test strips made according to the above-described embodiment. To show up the effect of the haematocrit mitigation strategy employed (which uses an algorithmic combination of the glucose reagent layer and ketone reagent layer each having different solvation and diffusion properties), a comparison ‘end current’ calibration is also shown. This is derived only from the current response of the glucose electrode only at 5 seconds, and demonstrates the high level of inherent haematocrit sensitivity present in the uncorrected response, and the measurement errors that could arise thereby.

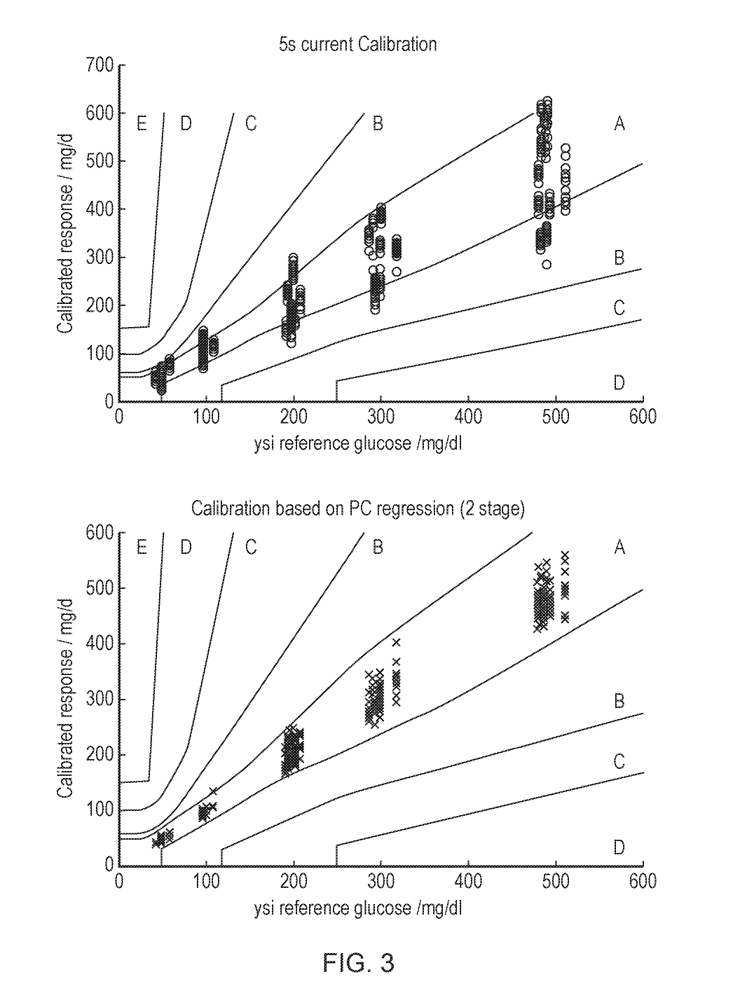

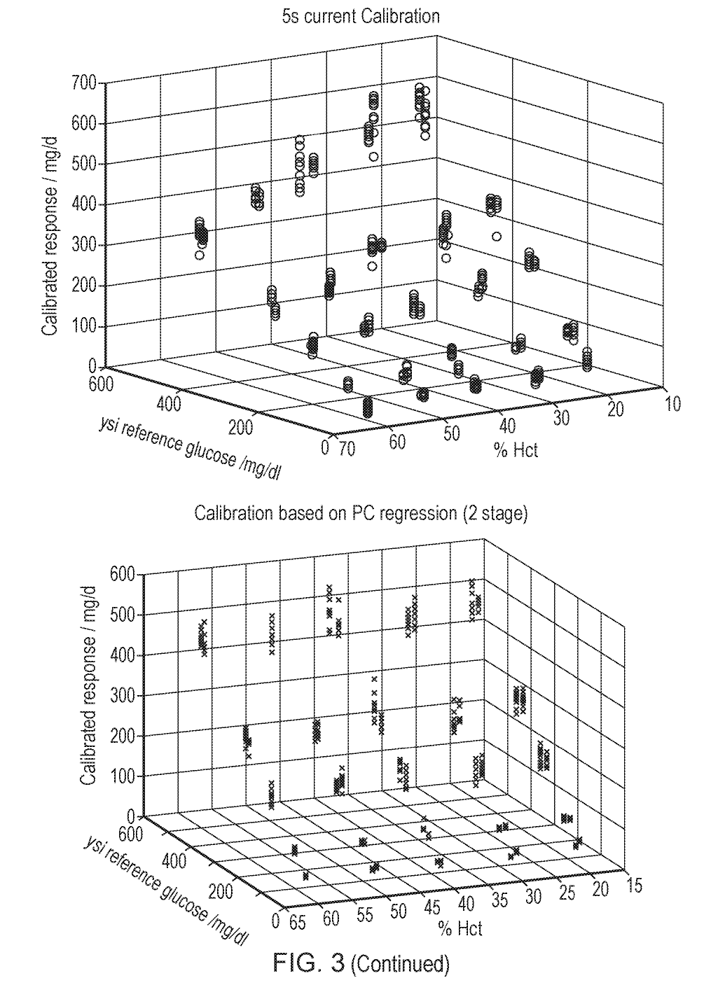

Measurement results for the two calibration methods are shown in FIG. 3. The improved accuracy and haematocrit mitigation possible with the PC algorithm is readily apparent by the reduced scatter and bias contributed by haematocrit.

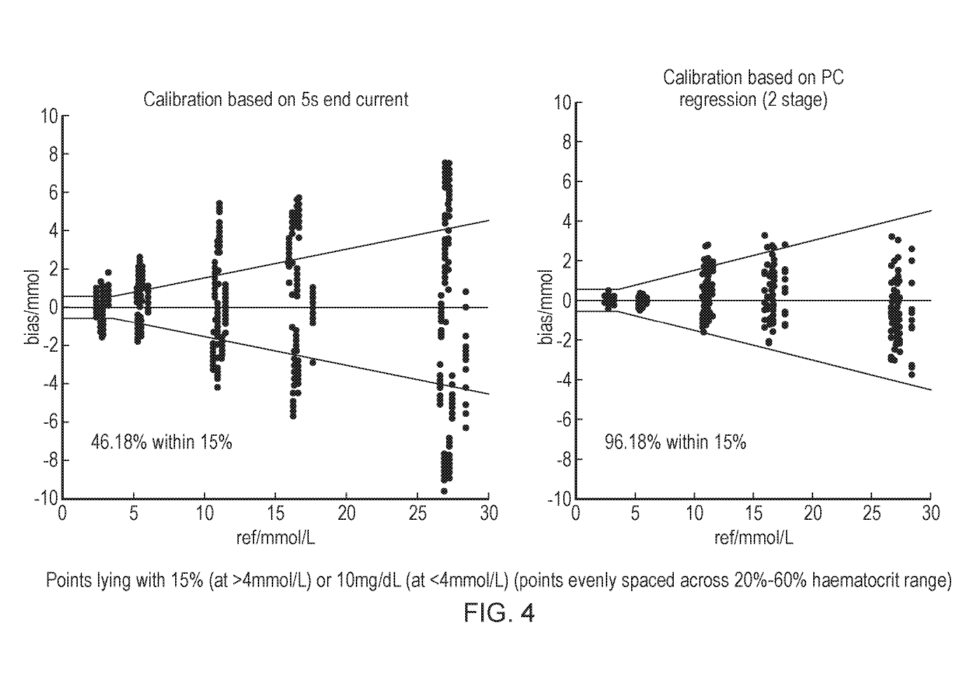

FIG. 4 shows the results bias (expressed in mmol/L) and demonstrates that the haematocrit mitigation strategy adopted has brought >95% the data to within 15% or 10 mg/dl of the reference value, this being achieved even when the sample distribution was evenly spread across the full limits of the haematocrit range (20-60%).

Up until this point, only the glucose response from electrodes 22a, corrected with electrode 24a, has been considered. A noteworthy element of this testing is that while the test strips of example 1 were being tested with samples in which the glucose and haematocrit levels were being adjusted, the concentration of beta-hydroxybutyrate (ketone) present in some of the samples used for testing was simultaneously being varied.

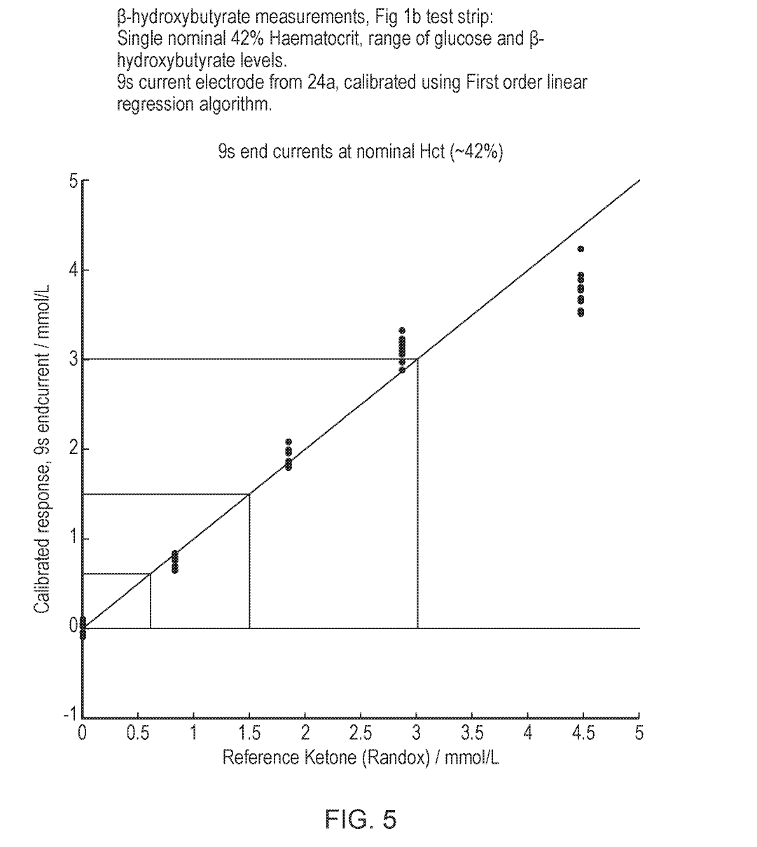

FIG. 5 shows the calibrated results of the blood β-hydroxybutyrate measurements simultaneously recorded from a subset of the same dual analyte test strips which produced the blood glucose measurements given in FIG. 3. The blood ketone levels and the blood glucose levels shown in the FIG. 5 dataset were separately varied, however the blood haematocrit remained fixed at a nominal level (42%). A simple first order linear regression model was used to convert the measured current values at 9 seconds from electrode 24a into corresponding β-hydroxybutyrate concentrations.

An improved ketone algorithm was derived using the same process as outlined in FIG. 9 but wherein ketone reference data (rather than the glucose reference data) was used for the construction of the model regression. Such a model derived using the same studentization coefficients and the same Principle Component coefficient matrix as employed within Algorithm 1 (for glucose measurement) was generated by stepwise regression. The model coefficients arrived at thereby and then subsequently used to produce a haematocrit corrected ketone measurements were as follows (designated Algorithm 1a):

| model_term | Coefficient |
|---|---|
| (Intercept) | 1.369 |
| x1 | 0.422 |
| x2 | −0.087 |
| x3 | −0.339 |
| x4 | −0.461 |
| x5 | 1.235 |
| x6 | 0.325 |
| x7 | −0.099 |
| x8 | −2.942 |
| x9 | −1.395 |
| x10 | 1.160 |
| x11 | −10.483 |
| x12 | −6.960 |
| x13 | 3.050 |
| x14 | −0.255 |
| x15 | 6.212 |
| x16 | −4.044 |
| x17 | 3.931 |
| x18 | 2.070 |
| x19 | 6.443 |
| x20 | −4.190 |
| x21 | 4.600 |
| x22 | −7.328 |
| x1:x3 | −0.094 |
| x1:x4 | −0.067 |
| x1:x5 | 0.290 |
| x1:x8 | −0.438 |
| x1:x9 | −0.651 |
| x1:x10 | −0.643 |
| x1:x11 | −2.670 |
| x1:x20 | −3.538 |
| x2:x3 | 0.057 |
| x2:x5 | −0.123 |
| x2:x6 | −0.156 |
| x2:x9 | 0.483 |
| x2:x10 | 0.814 |
| x2:x11 | 0.764 |
| x2:x18 | 3.575 |
| x2:x20 | 3.692 |
| x2:x22 | 3.612 |
| x3:x5 | −0.909 |
| x3:x7 | −1.548 |
| x3:x8 | 3.036 |
| x3:x9 | 1.826 |
| x3:x12 | 15.486 |
| x4:x5 | 2.413 |
| x4:x6 | 0.830 |
| x4:x8 | −4.214 |
| x4:x9 | −5.447 |
| x4:x11 | 24.025 |
| x4:x16 | 20.588 |
| x4:x18 | 21.033 |
| x4:x19 | −39.410 |
| x4:x21 | 37.265 |
| x4:x22 | −37.724 |
| x5:x7 | 8.250 |
| x5:x9 | −16.795 |
| x5:x13 | 33.545 |
| x5:x15 | −37.222 |
| x5:x19 | −55.643 |
| x6:x7 | −5.028 |
| x6:x9 | 10.612 |
| x6:x11 | −16.724 |
| x6:x12 | 42.661 |
| x7:x8 | 14.538 |
| x7:x9 | −31.283 |
| x7:x19 | 336.808 |
| x7:x21 | −221.406 |
| x8:x9 | 54.342 |
| x8:x21 | 399.280 |
| x9:x11 | 214.070 |
| x9:x14 | 265.531 |
| x11:x15 | −1166.536 |
| x12:x15 | −1124.430 |
| x12:x19 | −1699.346 |
| x15:x19 | 2137.908 |
| x16:x21 | −3412.518 |
| x17:x21 | 4726.595 |
| x18:x19 | 2751.107 |
| x18:x21 | −5389.044 |
| x18:x22 | −6585.457 |

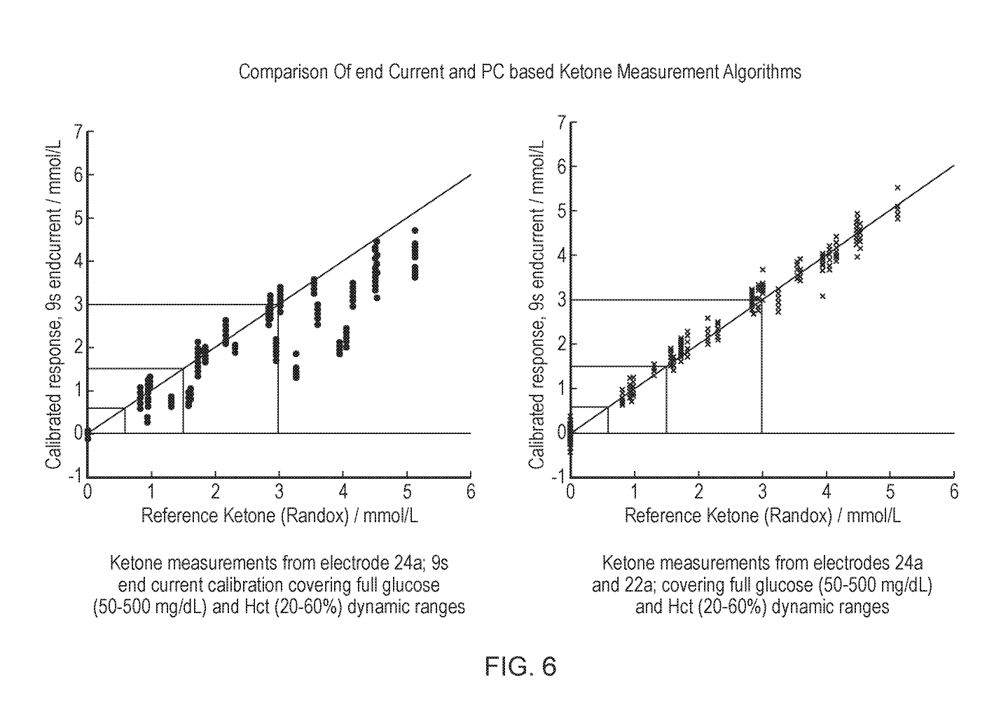

A comparison of the ketone measurement derived using Algorithm 1a above and a measurement derived using the same 9-second end current first order linear regression calibration algorithm as used in FIG. 5, but which covers the entire ketone, haematocrit and glucose measurement range is shown in FIG. 6.

Example 1 thereby demonstrates how the problems of inaccuracy within a multi-analyte measurement system which arise through ‘crosstalk’ effects and haematocrit sensitivity can simultaneously be addressed without the need for increased sample volumes, using a test strip that is compatible with low-cost high-volume manufacturing methodology and which only requires a small sample volume that is comparable with existing state of the art single analyte SMBG systems. Such a test strip is furthermore simultaneously capable of measurement accuracy that is in line with or exceeding current regulatory requirements for single-analyte SMBG test strips.

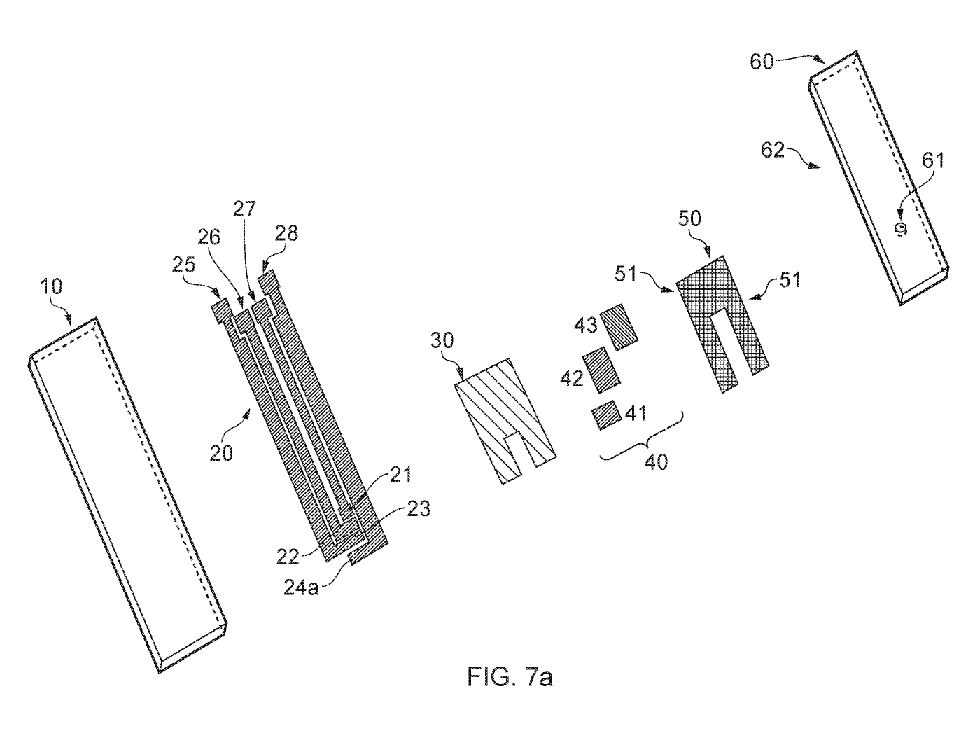

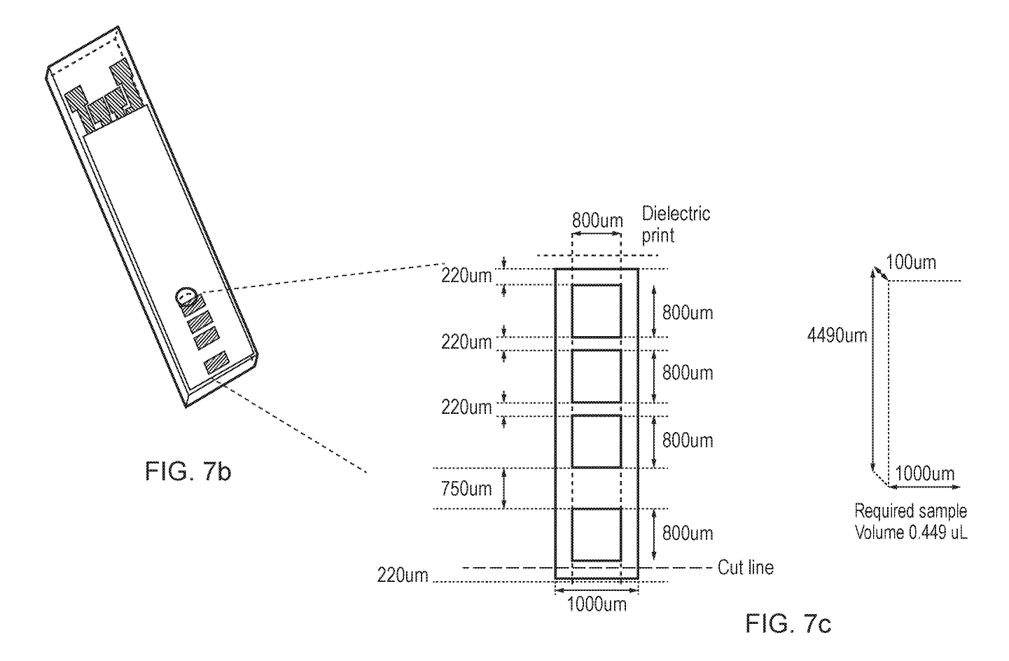

Now turning to FIGS. 7a-7c, there is shown a test strip according to a further embodiment of the invention. FIG. 7b shows a low-volume multi-analyte test strip. FIG. 7a shows the individual elements of the low-volume multi-analyte test strip, and the layers by which this is built up. FIG. 7c shows the typical dimensions of the main elements within the measurement volume of the multi-analyte test strip.

The positioning outlined successfully achieves a required sample volume of <0.45 μL whilst optimally allowing manufacturing tolerances that are consistent with high speed, high volume, low cost manufacturing processes, and an arrangements of functional components that enables accurate measurements predominantly free of crosstalk errors, while simultaneously enabling appropriate haematocrit mitigation strategies to be effected.

Referring to FIG. 7b, element 10 is a base substrate layer upon which is disposed a conductive electrode layer 20 which is patterned to form a plurality of working electrodes (21,23,24) and a reference/counter electrode (22) for interaction with a test sample and electrode contacts (25,26,27,28) for connection to a test meter. An optional dielectric layer (30) serves to precisely define the electrode areas exposed to the sample during measurement. Element 40 comprises a reagent layer, which is comprised of a plurality of reagent formulations (41,42,43) disposed so as to cover elements of the exposed electrodes (21,22,23,24). Element 50 shows a capillary spacer layer coated on both sides with an adhesive (51), and element 60 shows a cover layer with a hydrophilic coating on the underside (62) and a vent hole (61) through which air escapes when the capillary is filled with analyte containing sample.

In an illustrative use of this embodiment, reagent layer 41 is designed to be a moderate to fast dissolving and moderate to fast diffusing reagent composition which is electrochemically sensitive to beta-hydroxybutyrate, which completely covers working electrode 24 and does not impinge upon working electrodes 21 and 23, and reference/counter electrode 22. Reagent layer 43 is designed to be a rapidly dissolving and rapidly diffusing reagent composition which is electrochemically sensitive to beta-D-Glucose, and is designed to completely cover electrode 21 and to almost completely cover electrode 22 whilst avoiding impinging on working electrodes 23 and 24. Reagent layer 42 is designed to be a much more progressively dissolving and diffusing reagent composition, and is designed to completely cover electrode 23, which may incidentally cover a portion of working electrode 22, but which does not impinge upon working electrodes 21 or 24.

Whilst this layout and arrangement is particularly preferred with regard to the minimisation of carry-over crosstalk and diffusional crosstalk, other arrangements can also be effective for the multi-analyte measurement with appropriate care given to the separation and dissolution rates of the respective layers. Specifically, the location of the slow dissolution glucose reagent layer (42) between the reference/counter electrode (22) and the ‘ketone’ working electrode (24) is particularly beneficial.

It is apparent that this arrangement uses two different glucose measurement chemistries located in close proximity. This arrangement is adept at providing for a haematocrit mitigated measurement of the type described in GB1419799.0, filed 6 Nov. 2014, and incorporated herein by reference in its entirety. The presence of a rapidly solvating glucose measurement electrode maximises the underlying measurement precision that can be obtained from the system. When this response is taken in combination with the response signal from the more progressively solvating measurement electrode, the amount of ‘correction’ information that can be derived from which to account for sample variation effects (such as haematocrit variation) is simultaneously optimised. An algorithmic combination of the two signals thereby allows a highly precise haematocrit compensated measurement to be effected.

Additionally, due to the dual glucose measurement electrodes (21, 23), at least one of which is located at the furthest downstream locus on the sample fill path (21), the need for a separate fill-sufficiency detect electrode is obviated, by virtue of the fact that comparison of the signals from the two separate glucose electrodes for an expected level of consistency assures that adequate sample has been supplied to both electrodes (as well as improving the accuracy of the final glucose measurement). In this way, the required sample volume is minimised, with the multiple functions of haematocrit compensation, adjunctive glucose measurement confirmation, and fill assurance being accomplished by means of a single multifunctional additional design element, namely the slow diffusing glucose electrode. The multi-functionality of this single element thereby frees up sample volume from the design, within which the second analyte measurement components can be accommodated, allowing for the performance of accurate, haematocrit corrected multi-analyte measurements within a sample volume that is equivalent to that which might normally anyway be required by single analyte SMBG measurement systems.

### Manufacture of the Test Strip

Test strips were manufactured according to the design described above. The following method may be used to fabricate a plurality of test strips which may be referred to as a batch of test strips or a lot of test strips. However, it may be understood that the method may also be used to fabricate just one test strip. In other words, the following method may be used to manufacture one test strip or a plurality of test strips.

Specifically, sheets of substrate layer (10) comprised of 350 μm thick polyester were screen printed with a layer (20) of conductive Carbon ink and then a layer (30) of dielectric ink. Reagent layers (41,42,43) were screen printed on top using compositions comprised as follows.

Reagent layer (41) ink (covering electrode 24): 3 g of MBR hydroxyethyl cellulose was dissolved in 0.1M pH7.4 Tris buffer to a volume of 100 ml. To this was added 0.5 g Tween 20, 10 g of Ruthenium (III) Hexaammine Trichloride, 0.13 g of Flavin Mononucleotide, 1.3 g of NAD, 1 g of diaphorase and 1 g of beta-hydroxybutyrate dehydrogenase. A dry film weight of about 5 gsm was targeted. In this reagent layer, the low ratio of film formers to non-film formers and the relatively low film weight ensures a moderately rapid dissolution and solvation of the reagent layer in use.

Reagent layer (42) ink (covering electrode 23, impinging on 22): 25 g LR hydroxyethyl cellulose was dissolved in 100 ml 0.M pH 7.4 phosphate buffer to a volume of 100 ml. To this was added 1.5 g of fumed silica, 0.5 g of Tergitol NP9, 5 g of Potassium Hexacyanoferrate(III) and 2 g of Glucose Dehydrogenase FAD. A dry film weight of about 10 gsm was targeted. In this reagent layer, the high ratio of film formers to non-film formers and the relatively high film weight ensures a slower, more progressive dissolution and solvation of the reagent layer in use.

Reagent layer (43) ink (covering electrodes 21 and 22): 4 g of MBR hydroxyethyl cellulose was dissolved in 0.1M pH7.4 phosphate buffer to a volume of 100 ml. To this was added 1.5 g of fumed silica, 0.5 g of Tergitol NP9, 15 g of Potassium Hexacyanoferrate(III), and 2 g of FAD-Glucose Dehydrogenase. A dry film weight of about 6 gsm was targeted. In this reagent layer, the low ratio of film formers to non-film formers and the relatively low film weight ensures a moderately rapid dissolution and solvation of the reagent layer in use.

Capillary fill test strips were then formed by first cutting the printed sheets into rows, then laminating these with a multi-layered self-adhesive laminate (50,60). The laminated rows were then slit into individual test strips, and loading these into desiccated vials for storage. Dimensions were as outlined in FIG. 7c and the resulting test strips had a capillary sample volume of <0.5 μL.

### Testing of the Test Strips

The test strips from example 1 were tested as follows. The contact ends of the test strips (25,26,27,28) were first inserted into a test meter to make electrical connections with the electronics contained within. A potential was of 300 mV was thereby simultaneously applied across all three of working electrodes (21, 23, 24) relative to the RCESM (22). Test strips were filled with blood sample by capillary action, while current was measured simultaneously from each electrode. As soon as current exceeding 100 nA was detected from the downstream electrode (21), timing of the measurement began (t=0 seconds) and a time based response characteristic current was measured from each of the three electrode channels simultaneously. At t=85, the applied potential was reversed to −300 mV, and current measurements continued for a further 2s.

The measurement steps described above were completed for multiple electrodes using blood samples that had different, known concentrations of beta-D-glucose, beta-hydroxybutyrate (ketone) concentrations, and different, known sample haematocrit.

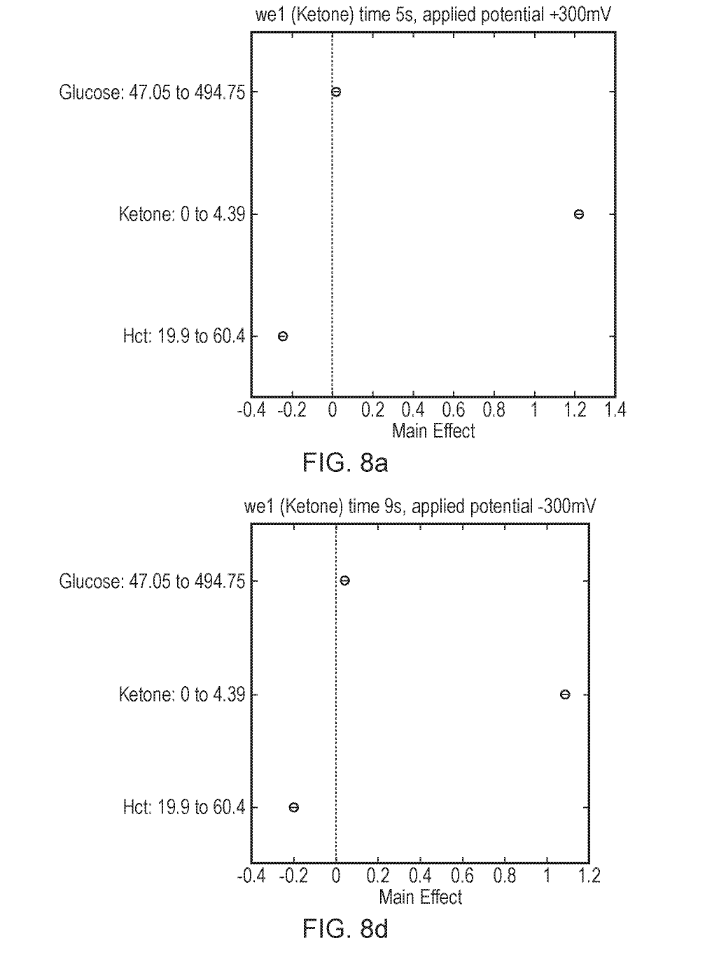

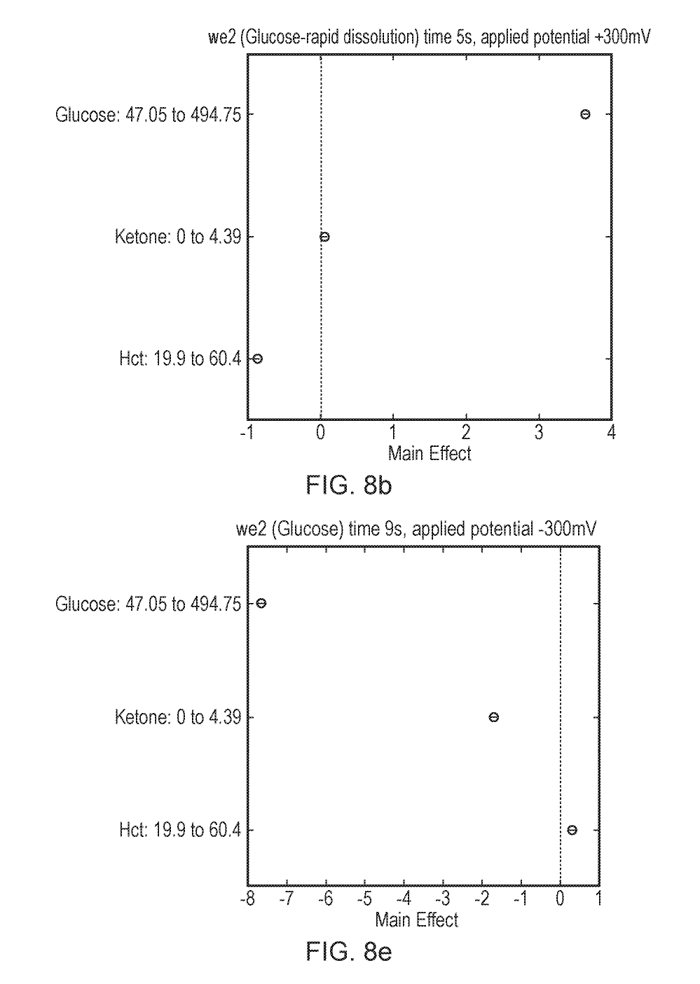

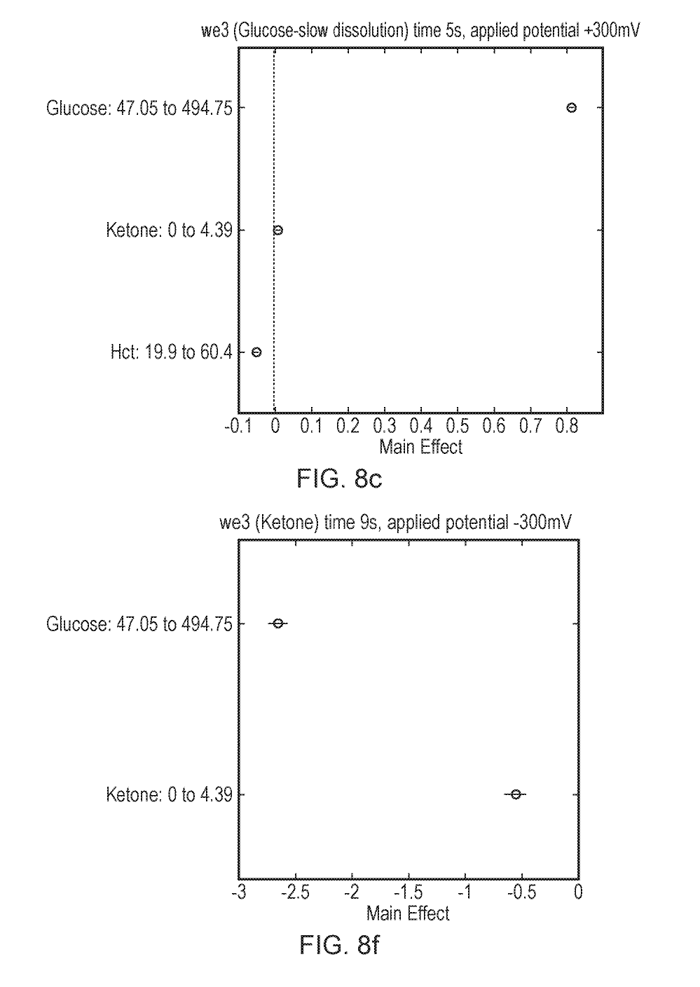

Analysis of variance (ANOVA) was performed for the current response characteristics at example sample times for each of the three electrodes, at time points before and after the potential was reversed, with the known glucose concentrations, ketone concentrations and sample haematocrits treated as dependent variables, and the resulting current treated as the response variable. The resulting main effects plots for the current responses are shown in FIGS. 8a-8f.

FIG. 8a shows the main effects plot for the ketone measurement electrode (24) at 5 seconds during the forward pulse. It shows a response that is highly sensitive to the ketone concentration, largely insensitive to the concentration of glucose (therefore ostensibly free of crosstalk) and somewhat sensitive to the effect of sample haematocrit.

FIG. 8b shows the main effects plot for the glucose measurement electrode (21) at 5 seconds during the forward pulse. It shows a response that is highly sensitive to the glucose concentration, largely insensitive to the concentration of ketone (therefore ostensibly free of crosstalk) and somewhat sensitive to the effect of sample haematocrit.

FIG. 8c shows the main effects plot for the other glucose measurement electrode (23) at 5 seconds during the forward pulse. It shows a response that is highly sensitive to the glucose concentration, largely insensitive to the concentration of ketone (therefore ostensibly free of crosstalk) and somewhat sensitive to the effect of sample haematocrit although relatively less so than either electrode 24 or electrode 21.

FIG. 8d shows the main effects plot for the ketone measurement electrode (24) at 9 seconds during the reverse pulse. It shows a response that is highly sensitive to the ketone concentration, with some sensitivity to the concentration of glucose (and therefore is suffering from electrolyte potential crosstalk) and somewhat sensitive to the effect of sample haematocrit.

FIG. 8e shows the main effects plot for the glucose measurement electrode (21) at 9 seconds during the reverse pulse. It shows a response that is highly sensitive to the glucose concentration, with significant sensitivity to the concentration of ketone (therefore ostensibly suffering from electrolyte potential crosstalk) and which is somewhat sensitive to the effect of sample haematocrit.

FIG. 8f shows the main effects plot for the glucose measurement electrode (23) at 9 seconds during the reverse pulse. It shows a response that is highly sensitive to the glucose concentration, with significant sensitivity to the concentration of ketone (and therefore is suffering from electrolyte potential crosstalk) and which is insensitive to the effect of sample haematocrit.

Thus, as can be seen, the test strip and testing configuration outlined above can simultaneously demonstrate the capability for (a) a substantially glucose concentration independent ketone measurement response, which is nevertheless haematocrit sensitive, (b) a substantially ketone concentration independent glucose measurement response which is nevertheless haematocrit sensitive, and (c) two simultaneously derived glucose dependent response signals characterised in that they have differentiated haematocrit sensitivities, by means of which response independent haematocrit information might be extracted. The response independent haematocrit information may be used to correct either or both of the substantially glucose independent ketone measurement signal of (a), and/or the substantially ketone independent glucose signals of (b).

This shows that the design configuration as employed has adequately mitigated ‘carry over’ and ‘diffusional’ crosstalk effects from the design, despite the very small sample volume achieved, and in a manner which is compatible with high volume low cost manufacturing methods.

It will also be understood that there are a great many sub-optimal alternate arrangements where the selection, relative placement, and separation of reagent layers could not achieve all of these elements simultaneously within such a confined sample volume, and also that larger sample volumes and greater element spacing would ordinarily be necessitated to accomplish all of these elements, in the normal course of events, were it not for the combination of fast and slow solvating and diffusing formulation elements.

It becomes apparent that to minimise the problems introduced by electrolyte potential crosstalk, maintaining the same, fixed potential at all measurement electrodes is the optimum strategy, and that potential ‘pulsing’ or potential reversal leads to the introduction of confounding of measurement signals across the different analyte electrodes, as evidence from a comparison of FIGS. 8a,b and c with the corresponding FIGS. 8d,e, and f. For this reason, prior art ‘pulsing’ strategies that have been applied to single analyte test systems with the intention of improving measurement performance are shown prima facie to be ineffective within multi-analyte systems, unless additional implementation methodologies and algorithms that specifically account for the electrolyte potential crosstalk effect are also introduced, such as that described previously using the test strip of FIG. 1a in combination with Algorithm 1 and/or Algorithm 2 and/or Algorithm 1a.

### Example 2

Test strips made according to FIG. 7a of the above embodiment were then tested for 7 seconds duration with +300 mV applied between all of the working electrodes (21,23,24) and the RCESM (22). A haematocrit corrected glucose response was derived from a ‘Principle Components’ calibration scheme derived according to the procedure outline in FIG. 9, which exploited the intentionally designed ‘differential’ solvation and diffusional properties of the two glucose inks to derive a haematocrit corrected response which simultaneously maximised the measurement precision.

In this example, a two-stage algorithm was used. The first algorithm (Algorithm 3) was trained using all reference glucose levels. The second algorithm (Algorithm 4) was trained only with reference glucose values below 200 mg/dL. When applying the algorithm to produce a calibrated response, Algorithm 3 was first used, and only where Algorithm 3 indicated a glucose result of 130 mg/dL or below was a result derived from Algorithm 4 substituted.

Both algorithms first used current measurements from the following time points (seconds) from the electrode response (23): 1.0 1.5 2.0 3.0 4.0 5.0 6.0 6.2 6.4 6.6 6.8, giving inputs 1-11. Current measurements from the following time points (seconds) from the electrode (21) response were then used: 3.0 4.0 5.0 5.3 5.6 5.9 6.2 6.5 6.8, giving inputs 12-20.

For Algorithm 3, the 20 raw inputs used the following mean and standard deviations:

| | elec- | | | |
|---|---|---|---|---|
| Algorithm_Input | trode | time_sec | mean_Amps | st_dev_Amps |
| 1 | 23 | 1 | 2.69125E−06 | 3.06568E−07 |
| 2 | 23 | 1.5 | 2.00807E−06 | 3.76109E−07 |
| 3 | 23 | 2 | 1.48443E−06 | 3.83509E−07 |
| 4 | 23 | 3 | 9.2785E−07 | 3.3624E−07 |
| 5 | 23 | 4 | 7.16792E−07 | 3.02786E−07 |
| 6 | 23 | 5 | 6.23747E−07 | 2.86169E−07 |
| 7 | 23 | 6 | 5.75101E−07 | 2.76079E−07 |
| 8 | 23 | 6.2 | 5.67971E−07 | 2.74478E−07 |
| 9 | 23 | 6.4 | 5.61637E−07 | 2.73203E−07 |
| 10 | 23 | 6.6 | 5.55886E−07 | 2.72124E−07 |
| 11 | 23 | 6.8 | 5.50589E−07 | 2.71153E−07 |
| 12 | 21 | 3 | 2.6137E−06 | 1.55113E−06 |
| 13 | 21 | 4 | 2.36596E−06 | 1.40156E−06 |
| 14 | 21 | 5 | 2.19974E−06 | 1.29798E−06 |
| 15 | 21 | 5.3 | 2.15927E−06 | 1.27363E−06 |
| 16 | 21 | 5.6 | 2.12228E−06 | 1.25126E−06 |
| 17 | 21 | 5.9 | 2.08809E−06 | 1.23109E−06 |
| 18 | 21 | 6.2 | 2.05678E−06 | 1.21259E−06 |
| 19 | 21 | 6.5 | 2.02739E−06 | 1.19506E−06 |
| 20 | 21 | 6.8 | 1.99994E−06 | 1.17923E−06 |

The coefficient matrix from which the model inputs (x1:x20) were derived by matrix multiplication of the 20 algorithm inputs for Algorithm 3 as follows:

| INPUT | 1 | 2 | 3 | 4 | 5 | 6 | 7 | 8 | 9 | 10 | 11 |
|---|---|---|---|---|---|---|---|---|---|---|---|
| 1 | 0.1976 | 0.3257 | 0.8807 | −0.1531 | −0.2225 | −0.0489 | 0.0597 | 0.0101 | 0.0074 | 0.0060 | 0.0040 |
| 2 | 0.2051 | 0.5609 | −0.0151 | 0.3208 | 0.5909 | 0.1308 | −0.3948 | 0.1215 | −0.0468 | −0.0100 | −0.0288 |
| 3 | 0.2086 | 0.4945 | −0.2712 | 0.2478 | −0.1630 | −0.1112 | 0.6060 | −0.3987 | 0.0727 | −0.0367 | 0.0707 |
| 4 | 0.2215 | 0.2642 | −0.2858 | −0.0190 | −0.5131 | −0.0494 | −0.0930 | 0.6688 | 0.0459 | 0.2309 | −0.1469 |
| 5 | 0.2270 | 0.1054 | −0.1718 | −0.1911 | −0.3580 | 0.0248 | −0.4469 | −0.3135 | −0.4486 | −0.4806 | 0.0939 |
| 6 | 0.2281 | 0.0257 | −0.1026 | −0.2648 | −0.0831 | 0.1104 | −0.3416 | −0.3104 | 0.7503 | 0.1496 | 0.1385 |
| 7 | 0.2282 | −0.0177 | −0.0640 | −0.2901 | 0.1241 | 0.0316 | 0.0103 | −0.1586 | −0.2325 | 0.5661 | 0.1610 |
| 8 | 0.2282 | −0.0233 | −0.0591 | −0.2911 | 0.1532 | 0.0130 | 0.0916 | −0.0412 | −0.2862 | 0.2784 | −0.0410 |
| 9 | 0.2282 | −0.0281 | −0.0562 | −0.2936 | 0.1748 | −0.0093 | 0.1482 | 0.0360 | −0.1627 | 0.0159 | −0.0811 |
| 10 | 0.2282 | −0.0325 | −0.0532 | −0.2972 | 0.2067 | −0.0464 | 0.1862 | 0.1544 | 0.0572 | −0.2522 | −0.1393 |
| 11 | 0.2281 | −0.0362 | −0.0509 | −0.2967 | 0.2247 | −0.0441 | 0.2029 | 0.2319 | 0.2387 | −0.4701 | −0.0401 |
| 12 | 0.2267 | −0.1647 | 0.0453 | 0.1712 | −0.0643 | 0.6857 | 0.1730 | 0.1807 | −0.0318 | −0.0775 | 0.5016 |
| 13 | 0.2267 | −0.1663 | 0.0454 | 0.1724 | −0.0694 | 0.3791 | 0.0553 | −0.0628 | −0.0298 | 0.0022 | −0.2684 |
| 14 | 0.2267 | −0.1662 | 0.0391 | 0.1736 | −0.0405 | 0.0911 | −0.0153 | −0.1058 | 0.0174 | 0.0243 | −0.3975 |
| 15 | 0.2267 | −0.1664 | 0.0375 | 0.1747 | −0.0256 | 0.0041 | −0.0275 | −0.0981 | 0.0254 | 0.0219 | −0.3079 |
| 16 | 0.2267 | −0.1666 | 0.0361 | 0.1752 | −0.0130 | −0.0815 | −0.0448 | −0.0625 | 0.0193 | 0.0257 | −0.2238 |
| 17 | 0.2267 | −0.1667 | 0.0349 | 0.1753 | 0.0005 | −0.1604 | −0.0415 | −0.0358 | 0.0120 | 0.0120 | −0.0638 |
| 18 | 0.2267 | −0.1668 | 0.0337 | 0.1751 | 0.0115 | −0.2404 | −0.0336 | 0.0092 | 0.0042 | 0.0178 | 0.0827 |
| 19 | 0.2267 | −0.1669 | 0.0322 | 0.1755 | 0.0250 | −0.3112 | −0.0411 | 0.0631 | −0.0022 | 0.0058 | 0.2520 |
| 20 | 0.2267 | −0.1669 | 0.0308 | 0.1767 | 0.0392 | −0.3719 | −0.0384 | 0.1088 | −0.0082 | −0.0286 | 0.4341 |

| INPUT | 12 | 13 | 14 | 15 | 16 | 17 | 18 | 19 | 20 |
|---|---|---|---|---|---|---|---|---|---|
| 1 | 0.0009 | −0.0011 | −0.0001 | −0.0031 | −0.0002 | −0.0015 | 0.0006 | 0.0003 | 0.0004 |
| 2 | −0.0010 | 0.0047 | 0.0045 | 0.0048 | 0.0015 | 0.0048 | −0.0018 | −0.0040 | −0.0020 |
| 3 | −0.0033 | −0.0043 | −0.0097 | −0.0019 | −0.0015 | −0.0040 | 0.0005 | 0.0095 | 0.0027 |
| 4 | 0.0076 | −0.0050 | 0.0155 | 0.0013 | −0.0005 | −0.0031 | 0.0052 | −0.0111 | −0.0002 |
| 5 | 0.0567 | 0.0051 | −0.0080 | −0.0131 | −0.0036 | 0.0054 | −0.0107 | 0.0026 | −0.0015 |
| 6 | −0.1696 | 0.0196 | −0.0189 | 0.0198 | 0.0068 | 0.0045 | 0.0068 | 0.0026 | −0.0019 |
| 7 | 0.6242 | 0.0968 | 0.1331 | 0.0136 | −0.0063 | −0.0071 | −0.0140 | 0.0047 | −0.0098 |
| 8 | −0.4357 | −0.4698 | −0.5022 | −0.0143 | 0.0074 | 0.0001 | 0.0225 | −0.0025 | 0.0276 |
| 9 | −0.4890 | 0.2044 | 0.6994 | −0.0145 | 0.0029 | 0.0023 | −0.0028 | 0.0034 | −0.0162 |
| 10 | 0.0516 | 0.6677 | −0.4618 | −0.0387 | 0.0335 | −0.0278 | 0.0226 | −0.0117 | −0.0165 |
| 11 | 0.3586 | −0.5217 | 0.1499 | 0.0481 | −0.0405 | 0.0252 | −0.0285 | 0.0062 | 0.0175 |
| 12 | −0.0469 | 0.0243 | −0.0195 | −0.3009 | −0.0236 | −0.0012 | −0.0113 | 0.0139 | −0.0042 |
| 13 | 0.0114 | 0.0310 | −0.0119 | 0.8014 | −0.1110 | −0.0073 | 0.0084 | −0.0648 | 0.0244 |
| 14 | 0.0765 | −0.0451 | 0.0280 | −0.2119 | 0.5590 | 0.4121 | 0.1823 | 0.3588 | −0.1063 |
| 15 | 0.0499 | −0.0484 | 0.0310 | −0.2624 | 0.2635 | −0.4309 | −0.3405 | −0.5659 | 0.1269 |
| 16 | 0.0189 | −0.0165 | −0.0005 | −0.1941 | −0.4707 | −0.4506 | −0.0974 | 0.5752 | −0.1475 |
| 17 | 0.0255 | 0.0039 | 0.0252 | −0.1753 | −0.3739 | 0.1476 | 0.6818 | −0.2995 | 0.3280 |
| 18 | −0.0314 | 0.0087 | −0.0327 | −0.0291 | −0.2831 | 0.4686 | −0.3129 | −0.2745 | −0.5888 |
| 19 | −0.0512 | 0.0565 | −0.0195 | 0.1121 | 0.0418 | 0.2388 | −0.4229 | 0.2212 | 0.6457 |
| 20 | −0.0535 | −0.0110 | −0.0018 | 0.2582 | 0.3987 | −0.3761 | 0.3121 | 0.0357 | −0.2781 |

The regression equation coefficients, by which the measured result was derived from the 20 model inputs (x1:x2) for Algorithm 3 were as follows:

| model_term | Coefficient |
|---|---|
| (Intercept) | 220.98 |
| x1 | 35.75 |
| x2 | 5.97 |
| x3 | −33.55 |
| x4 | 3.00 |
| x5 | 93.61 |
| x6 | −93.04 |
| x7 | 82.26 |
| x8 | 221.85 |
| x9 | 31.04 |
| x10 | −185.37 |
| x11 | 559.05 |
| x12 | 126.67 |
| x13 | −158.86 |
| x14 | 29.70 |
| x15 | −245.41 |
| x16 | 1371.40 |
| x17 | 1002.01 |
| x18 | 987.67 |
| x19 | −434.22 |
| x20 | −770.72 |
| x1:x2 | 5.65 |
| x1:x3 | −8.00 |
| x1:x4 | −4.97 |
| x1:x6 | −24.57 |
| x1:x7 | −20.29 |
| x1:x15 | −240.95 |
| x1:x17 | −273.13 |
| x1:x18 | 325.39 |
| x1:x20 | −416.80 |
| x2:x5 | −44.27 |
| x2:x10 | 509.57 |
| x2:x11 | −457.38 |
| x2:x14 | 429.86 |
| x2:x16 | −1663.69 |
| x3:x8 | −372.49 |
| x3:x11 | −695.54 |
| x3:x12 | 1007.79 |
| x3:x18 | 3833.75 |
| x4:x11 | −2633.29 |
| x4:x12 | 1356.72 |
| x4:x15 | −2923.42 |
| x4:x19 | 9069.74 |
| x5:x6 | −987.02 |
| x5:x8 | 5653.15 |
| x5:x10 | −3207.78 |
| x5:x13 | −7957.13 |
| x6:x9 | 6280.62 |
| x6:x18 | −64861.48 |
| x8:x15 | −42745.83 |
| x8:x16 | 86654.53 |
| x9:x19 | 126356.26 |
| x10:x18 | 156094.05 |
| x11:x19 | −311971.63 |
| x12:x13 | 81076.72 |
| x15:x19 | −585291.34 |
| x18:x20 | 1784626.99 |

For Algorithm 4, the 20 raw inputs used the following mean and standard deviations:

| | elec- | | | |
|---|---|---|---|---|
| Algorithm_Input | trode | time_sec | mean_Amps | st_dev_Amps |
| 1 | 23 | 1.0 | 2.5972E−06 | 1.8325E−07 |
| 2 | 23 | 1.5 | 1.8310E−06 | 2.3448E−07 |
| 3 | 23 | 2.0 | 1.2739E−06 | 2.2666E−07 |
| 4 | 23 | 3.0 | 7.0738E−07 | 1.4874E−07 |
| 5 | 23 | 4.0 | 5.0708E−07 | 1.1515E−07 |
| 6 | 23 | 5.0 | 4.2206E−07 | 1.0451E−07 |
| 7 | 23 | 6.0 | 3.7965E−07 | 1.0132E−07 |
| 8 | 23 | 6.2 | 3.7336E−07 | 1.0089E−07 |
| 9 | 23 | 6.4 | 3.6779E−07 | 1.0060E−07 |
| 10 | 23 | 6.6 | 3.6309E−07 | 1.0058E−07 |
| 11 | 23 | 6.8 | 3.5765E−07 | 1.0024E−07 |
| 12 | 21 | 3.0 | 1.5340E−06 | 5.9041E−07 |
| 13 | 21 | 4.0 | 1.3894E−06 | 5.4476E−07 |
| 14 | 21 | 5.0 | 1.2926E−06 | 5.1092E−07 |
| 15 | 21 | 5.3 | 1.2688E−06 | 5.0347E−07 |
| 16 | 21 | 5.6 | 1.2466E−06 | 4.9727E−07 |
| 17 | 21 | 5.9 | 1.2263E−06 | 4.9158E−07 |
| 18 | 21 | 6.2 | 1.2072E−06 | 4.8628E−07 |
| 19 | 21 | 6.5 | 1.1899E−06 | 4.8148E−07 |
| 20 | 21 | 6.8 | 1.1735E−06 | 4.7673E−07 |

The coefficient matrix from which the model inputs (x1:x20) were derived by matrix multiplication of the 20 algorithm inputs for Algorithm 4 was as follows:

| INPUT | 1 | 2 | 3 | 4 | 5 | 6 | 7 | 8 | 9 | 10 | 11 |
|---|---|---|---|---|---|---|---|---|---|---|---|
| 1 | 0.0908 | 0.2777 | 0.9307 | 0.0147 | −0.2076 | −0.0513 | −0.0345 | −0.0060 | 0.0278 | −0.0187 | −0.0033 |
| 2 | 0.0995 | 0.5166 | −0.0025 | 0.3686 | 0.6220 | 0.2058 | 0.1794 | −0.0551 | −0.3475 | 0.0102 | 0.0117 |
| 3 | 0.1159 | 0.4928 | −0.2157 | 0.2873 | −0.0855 | −0.2308 | −0.1533 | 0.2619 | 0.6550 | 0.1554 | −0.0107 |
| 4 | 0.1805 | 0.3770 | −0.2526 | 0.0537 | −0.4685 | −0.1281 | −0.2263 | −0.4113 | −0.2924 | −0.4279 | −0.0227 |
| 5 | 0.2282 | 0.2179 | −0.1355 | −0.1759 | −0.4254 | 0.2858 | 0.1150 | 0.2953 | −0.3694 | 0.5682 | 0.0776 |
| 6 | 0.2425 | 0.1130 | −0.0492 | −0.2644 | −0.0796 | 0.1979 | 0.7467 | −0.2422 | 0.3343 | −0.2164 | −0.1213 |
| 7 | 0.2464 | 0.0520 | −0.0103 | −0.2868 | 0.1210 | 0.0938 | −0.0830 | 0.4599 | 0.0006 | −0.4584 | 0.6098 |
| 8 | 0.2468 | 0.0427 | −0.0063 | −0.2845 | 0.1476 | 0.0502 | −0.2526 | 0.2225 | −0.0109 | −0.1562 | −0.4346 |
| 9 | 0.2470 | 0.0350 | −0.0003 | −0.2854 | 0.1729 | −0.0188 | −0.1566 | 0.1364 | −0.0414 | −0.0070 | −0.5569 |
| 10 | 0.2471 | 0.0313 | 0.0029 | −0.2841 | 0.2024 | −0.1750 | −0.0906 | −0.2652 | 0.0862 | 0.2076 | 0.2113 |
| 11 | 0.2471 | 0.0263 | 0.0042 | −0.2853 | 0.2062 | −0.2666 | −0.1372 | −0.4050 | 0.0385 | 0.3494 | 0.2351 |
| 12 | 0.2393 | −0.1567 | 0.0269 | 0.1874 | 0.0033 | 0.5496 | −0.3747 | −0.2736 | 0.1652 | −0.0463 | 0.0197 |
| 13 | 0.2398 | −0.1550 | 0.0257 | 0.1761 | −0.0326 | 0.3372 | −0.1157 | −0.0692 | 0.1417 | 0.0507 | 0.0669 |
| 14 | 0.2403 | −0.1516 | 0.0189 | 0.1715 | −0.0420 | 0.0846 | 0.0569 | 0.0336 | 0.0577 | 0.0782 | 0.0109 |
| 15 | 0.2405 | −0.1504 | 0.0145 | 0.1707 | −0.0416 | −0.0008 | 0.0641 | 0.0487 | 0.0345 | 0.0427 | −0.0096 |
| 16 | 0.2406 | −0.1496 | 0.0131 | 0.1711 | −0.0293 | −0.0616 | 0.0864 | 0.0596 | −0.0083 | 0.0091 | −0.0236 |
| 17 | 0.2407 | −0.1484 | 0.0109 | 0.1700 | −0.0219 | −0.1308 | 0.0970 | 0.0585 | −0.0441 | 0.0209 | −0.0376 |
| 18 | 0.2408 | −0.1475 | 0.0093 | 0.1709 | −0.0125 | −0.2010 | 0.0993 | 0.0478 | −0.0632 | −0.0307 | −0.0073 |
| 19 | 0.2409 | −0.1471 | 0.0068 | 0.1712 | −0.0039 | −0.2645 | 0.0905 | 0.0716 | −0.1413 | −0.0701 | 0.0046 |
| 20 | 0.2410 | −0.1460 | 0.0055 | 0.1722 | 0.0056 | −0.3111 | 0.0530 | 0.0419 | −0.1649 | −0.0610 | −0.0217 |

| INPUT | 12 | 13 | 14 | 15 | 16 | 17 | 18 | 19 | 20 |
|---|---|---|---|---|---|---|---|---|---|
| 1 | −0.0032 | −0.0020 | 0.0051 | −0.0008 | −0.0006 | 0.0001 | 0.0004 | 0.0010 | 0.0008 |
| 2 | 0.0169 | 0.0204 | −0.0328 | −0.0015 | 0.0008 | 0.0028 | −0.0090 | −0.0047 | −0.0022 |
| 3 | −0.0025 | −0.0384 | 0.0832 | 0.0033 | −0.0019 | −0.0019 | 0.0167 | 0.0033 | 0.0014 |
| 4 | −0.0386 | 0.0213 | −0.1761 | −0.0017 | 0.0011 | 0.0020 | −0.0092 | 0.0082 | 0.0012 |
| 5 | 0.0095 | 0.0085 | 0.1485 | −0.0060 | −0.0099 | −0.0088 | −0.0087 | −0.0135 | 0.0002 |
| 6 | 0.0582 | −0.0334 | 0.1231 | 0.0245 | 0.0179 | 0.0065 | 0.0208 | −0.0002 | −0.0050 |
| 7 | −0.0372 | −0.1522 | −0.0394 | −0.0484 | 0.0322 | 0.0125 | −0.0227 | 0.0118 | 0.0105 |
| 8 | 0.5244 | 0.4668 | 0.0350 | 0.0759 | −0.0080 | −0.0614 | 0.0348 | 0.0191 | 0.0072 |
| 9 | −0.4733 | −0.4774 | −0.1179 | −0.0365 | −0.0003 | 0.0358 | −0.0250 | −0.0239 | −0.0365 |
| 10 | −0.5185 | 0.5791 | 0.0634 | −0.0126 | −0.0380 | 0.0437 | −0.0012 | −0.0088 | 0.0132 |
| 11 | 0.4578 | −0.3990 | −0.1079 | 0.0018 | 0.0039 | −0.0301 | 0.0054 | 0.0076 | 0.0095 |
| 12 | −0.0039 | −0.1012 | 0.4683 | −0.3211 | −0.0232 | 0.0230 | 0.0070 | 0.0240 | 0.0264 |
| 13 | −0.0658 | −0.0156 | −0.2770 | 0.7932 | −0.0389 | −0.0781 | −0.0867 | −0.0152 | −0.0298 |
| 14 | −0.0265 | 0.0844 | −0.4486 | −0.2997 | 0.3273 | −0.2951 | 0.5526 | 0.1234 | −0.2300 |
| 15 | 0.1034 | 0.0873 | −0.2922 | −0.2097 | −0.0441 | 0.6869 | −0.2690 | −0.2659 | −0.3382 |
| 16 | 0.0091 | 0.0216 | −0.1893 | −0.1485 | −0.2117 | −0.0671 | 0.0603 | −0.4002 | 0.7762 |
| 17 | 0.0028 | 0.0214 | −0.0542 | −0.0727 | 0.0756 | 0.0956 | −0.3377 | 0.8032 | 0.2722 |
| 18 | −0.0057 | 0.0171 | 0.1019 | −0.1251 | −0.0985 | −0.6158 | −0.5157 | −0.2168 | −0.3239 |
| 19 | 0.0000 | −0.0652 | 0.2674 | 0.1365 | −0.6349 | 0.0932 | 0.4594 | 0.1448 | −0.2111 |
| 20 | −0.0083 | −0.0474 | 0.4336 | 0.2490 | 0.6500 | 0.1572 | 0.1288 | −0.1965 | 0.0582 |

The regression equation coefficients, by which the measured result was derived from the 20 model inputs (x1:x2) for Algorithm 4 were as follows:

| model_term | Coefficient |
|---|---|
| (Intercept) | 106.12 |
| x1 | 13.99 |
| x2 | −0.84 |
| x3 | −6.02 |
| x4 | 9.06 |
| x5 | 34.86 |
| x6 | −60.17 |
| x7 | −47.85 |
| x9 | −53.62 |
| x10 | −45.75 |
| x11 | −12.07 |
| x12 | −58.35 |
| x13 | −39.58 |
| x14 | 281.50 |
| x15 | 294.94 |
| x16 | 43.64 |
| x17 | −189.85 |
| x18 | 84.23 |
| x19 | −24.65 |
| x20 | 53.80 |
| x1:x2 | 0.71 |
| x2:x3 | −0.98 |
| x2:x4 | 1.37 |
| x2:x11 | −27.44 |
| x2:x12 | 45.45 |
| x3:x12 | 104.01 |
| x3:x15 | −248.58 |
| x4:x15 | 244.69 |
| x4:x20 | 327.27 |
| x5:x9 | −419.57 |
| x5:x15 | 924.55 |
| x7:x9 | 745.81 |
| x7:x14 | −1642.67 |
| x9:x14 | −1766.59 |
| x9:x16 | 3723.97 |
| x10:x18 | −5750.80 |
| x10:x20 | −8761.22 |
| x12:x19 | −5908.87 |
| x14:x18 | 19604.47 |
| x15:x18 | 19341.68 |
| x15:x20 | 38606.51 |
| x18:x19 | −63320.07 |

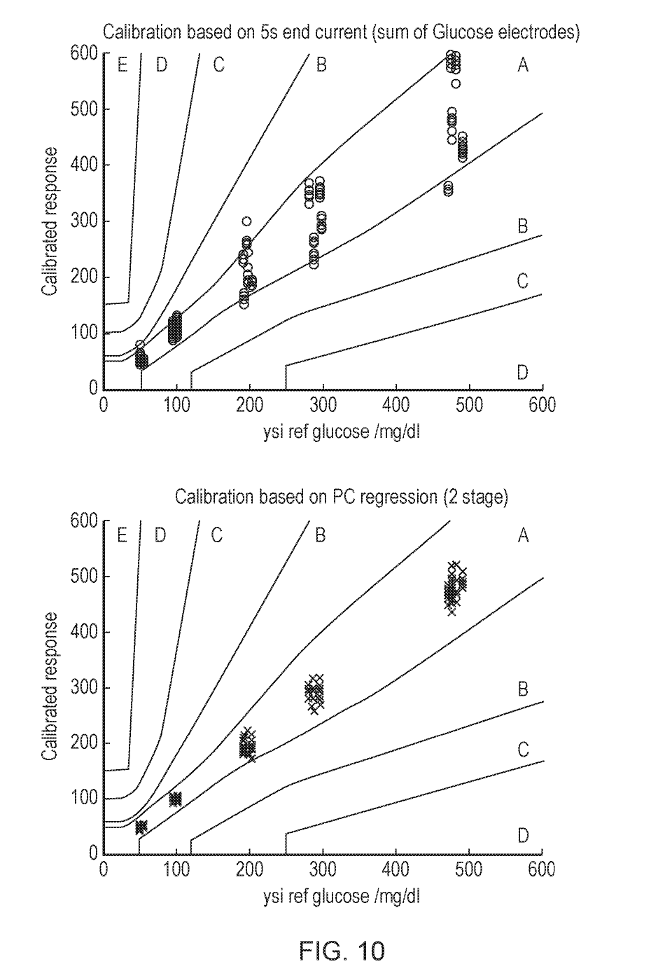

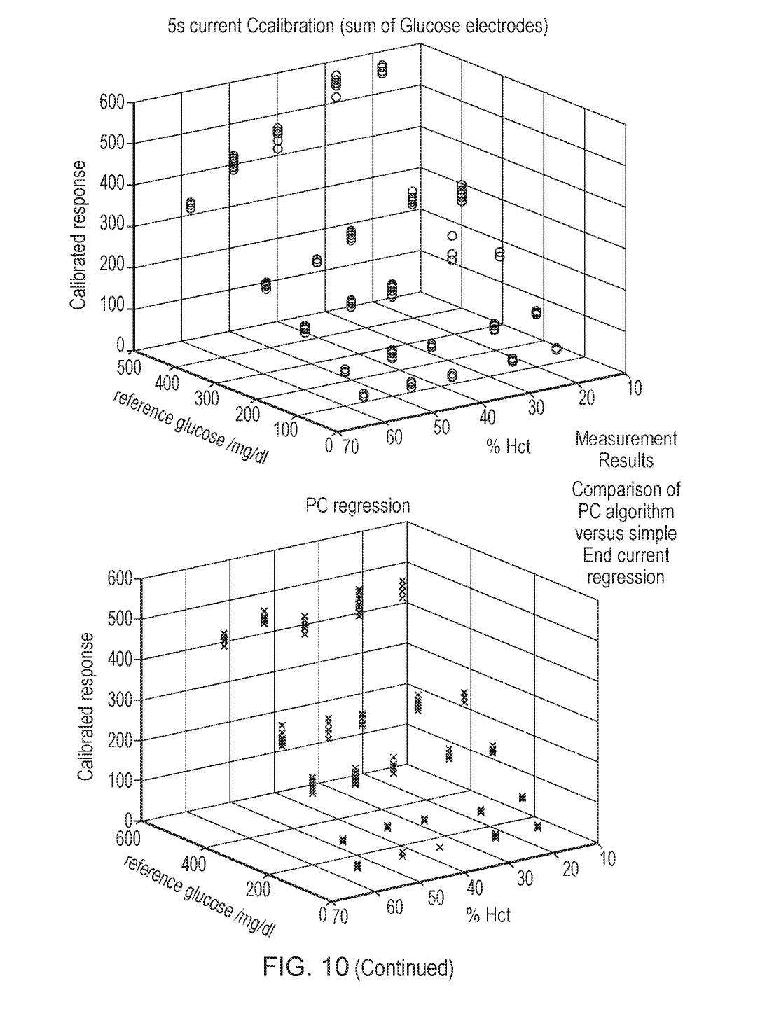

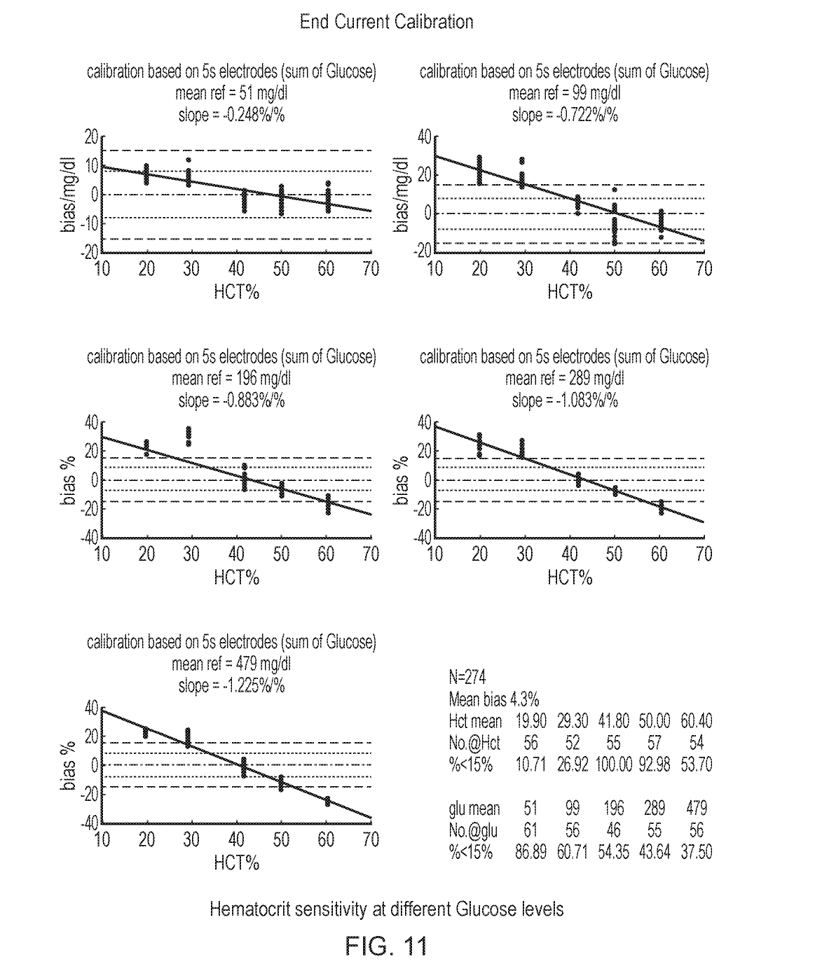

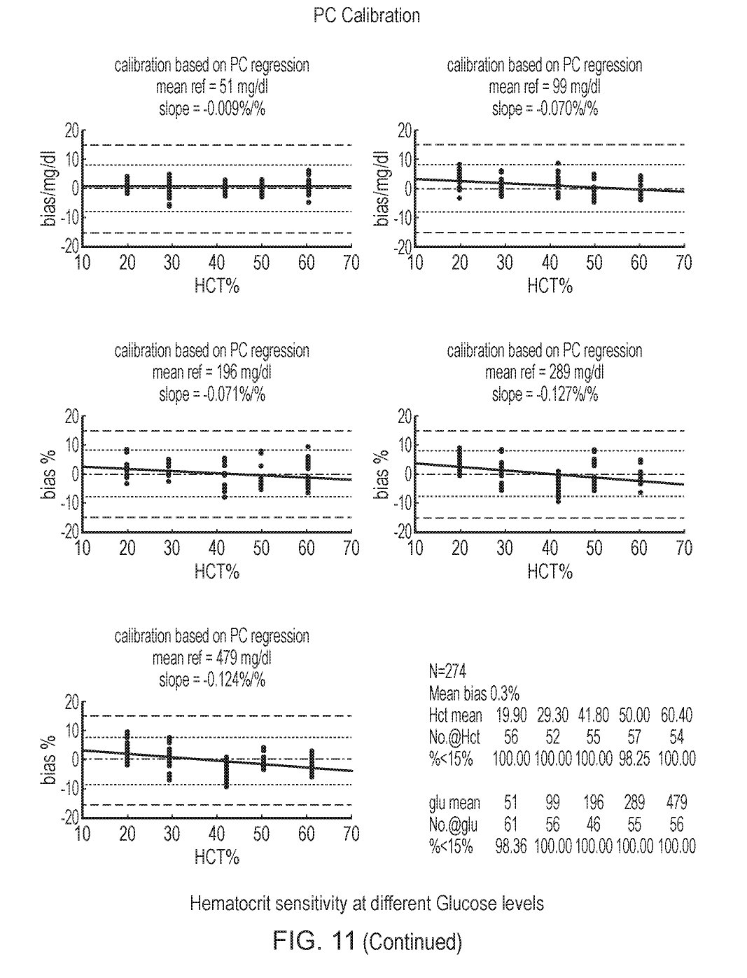

The algorithms described above were used to produce calibrated measurements from tests carried out using the test strips described above. To illustrate the effect of the haematocrit mitigation strategy employed (using an algorithmic combination of the two glucose reagent layers with different solvation and diffusion properties), a comparison ‘end current’ calibration is also shown. This is derived only from the sum of current response of the two glucose electrodes between 5 and 6 seconds, and demonstrates the inherent haematocrit sensitivity of the uncorrected response, and the errors that can arise thereby. Measurement results for the two calibration methods are shown in FIG. 10. FIG. 11 shows bias plots demonstrating the sensitivity to haematocrit observed at each of the glucose levels tested, comparing the end current derived measurement with the PC algorithm approach.

This demonstrates that the PC algorithm approach has largely eliminated any haematocrit sensitivity from the measured result, as can be seen by the reduced scatter and bias contributed by haematocrit. Furthermore, it can be seen that when the algorithm was employed, more than 95% of measured results lay within 15% of the reference value for every individual glucose and haematocrit combination assessed, whereas for the end current calibration, only the nominal 42% haematocrit level achieved this accuracy standard. This demonstrates the capability of the test strips of FIGS. 7a-7c to produce highly accurate, haematocrit compensated measurements of blood glucose using the methodology described.

Up until this point, only the glucose response from electrodes 21 and 23 has been considered. A noteworthy element of this testing is that—while the test strips of FIGS. 7a-7c were being tested with samples in which the glucose and haematocrit levels were being adjusted, the concentration of beta-hydroxybutyrate present in some of the samples used for testing was simultaneously being varied.

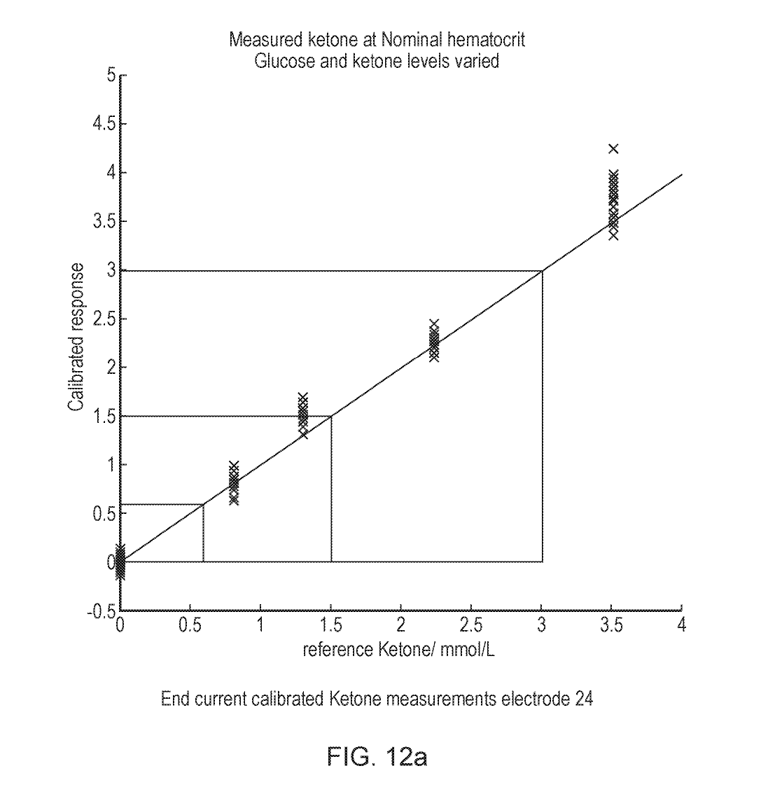

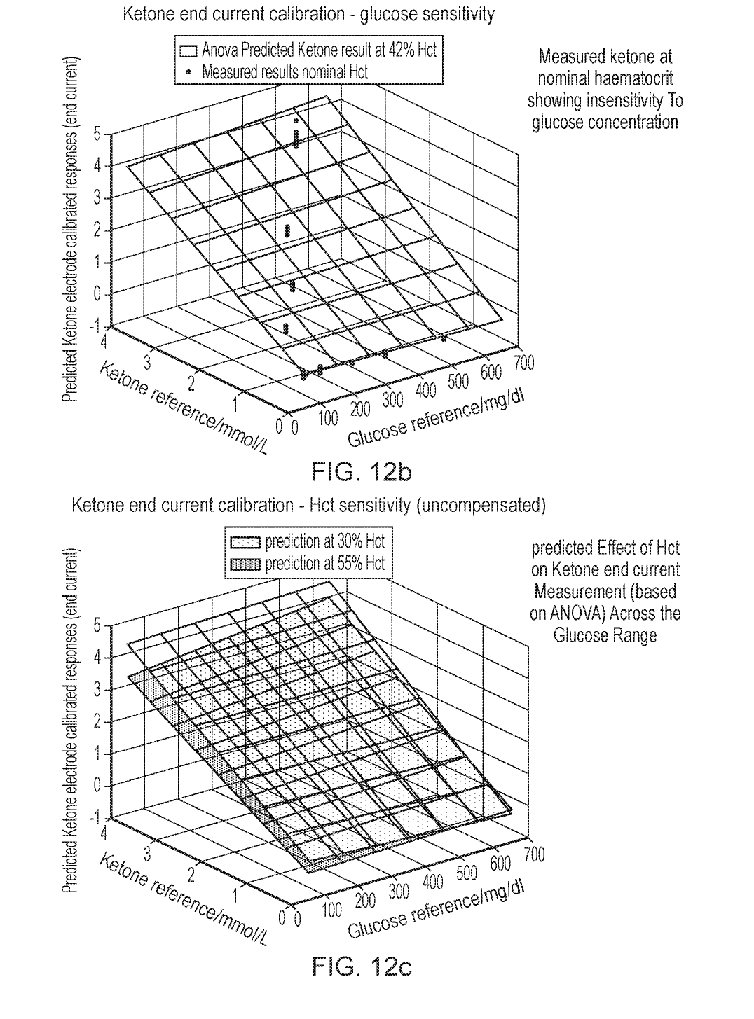

FIGS. 12a-12c show the corresponding ketone measurements from electrode 24 for the tests in example 2 which were performed at nominal haematocrit (42%).

FIG. 12a shows the result of using the average of all currents from electrode 24 between 5 and 6 seconds calibrated using a first order linear regression to produce a ketone measurement from blood samples tested. The measured ketone results were obtained simultaneously to the measured glucose results shown previously, derived from the same individual test strips and at the same time using the same single sample drop.

FIG. 12b shows measurement data from the same ketone levels at different glucose levels, demonstrating the absence of crosstalk (from changes in glucose level) in the ketone measurements produced.

FIG. 12c shows the modelled impact of changes in haematocrit (assessed using ANOVA) on the ketone measurements. FIG. 12c shows a fairly modest impact of haematocrit on the ketone measurement. However, this measurement can be further enhanced in that regard by exploiting the haematocrit information that can be derived from the glucose measurement effected previously from the ‘differential’ solvation glucose measurement electrodes.

In this case, a further PC algorithm is derived using the same raw inputs and coefficient matrices as used in Algorithms 3 and 4, and using the scheme outlined in FIG. 9, but in this instance instead of training the algorithm model with reference glucose data, the algorithm was instead trained using the known haematocrit values of the samples used in the training set. The resulting model parameters (shown below—Algorithms 3a and 4a) could thereby be used along with the signals from electrodes 21 and 23 to derive a useful estimate of the actual haematocrit value of each of the samples.

| | Hct fit |
|---|---|
| model_term | Coefficient |
| Alg 3a | |
| (Intercept) | 41.39 |
| x1 | −0.60 |
| x2 | 13.13 |
| x3 | −9.44 |
| x4 | −13.71 |
| x5 | 22.25 |
| x6 | −70.08 |
| x7 | 16.58 |
| x8 | −13.65 |
| x9 | 4.28 |
| x10 | −32.54 |
| x11 | −30.06 |
| x12 | 0.68 |
| x13 | −59.68 |
| x14 | 7.21 |
| x15 | −127.87 |
| x16 | 105.20 |
| x17 | −77.69 |
| x19 | 176.71 |
| x20 | −409.62 |
| x1:x4 | 1.17 |
| x1:x5 | −4.92 |
| x1:x6 | 5.30 |
| x1:x7 | 9.84 |
| x1:x12 | 40.30 |
| x2:x4 | 5.28 |
| x2:x5 | −11.62 |
| x2:x10 | 117.50 |
| x2:x12 | −122.24 |
| x3:x6 | −77.63 |
| x3:x8 | −155.38 |
| x3:x11 | −326.30 |
| x4:x10 | −244.80 |
| x4:x12 | 382.61 |
| x4:x15 | −833.00 |
| x4:x19 | 2255.18 |
| x5:x8 | 1345.89 |
| x8:x10 | −3092.96 |
| x8:x12 | 7554.59 |
| x8:x15 | −10018.76 |
| x9:x15 | −21146.97 |
| x11:x16 | 56657.29 |
| x13:x15 | 59305.71 |
| x13:x19 | 116779.32 |
| x14:x15 | −33099.30 |
| x17:x19 | −322288.31 |
| Alg 4a | |
| (Intercept) | 41.7076503 |
| x2 | 5.52237737 |
| x3 | −4.1223843 |
| x4 | −2.4813965 |
| x5 | 10.4462501 |
| x6 | −18.405242 |
| x7 | 48.8486199 |
| x8 | 20.386897 |
| x10 | 9.00816888 |
| x11 | −32.008407 |
| x13 | 6.11626678 |
| x14 | −29.974626 |
| x15 | 81.8774491 |
| x17 | −13.822853 |
| x18 | 92.5664773 |
| x20 | 121.726933 |
| x2:x3 | −0.3641212 |
| x2:x10 | −18.579112 |
| x3:x5 | −11.116744 |
| x3:x7 | 58.4798133 |
| x3:x8 | 51.910042 |
| x3:x11 | −47.643905 |
| x4:x7 | −28.346556 |
| x5:x8 | −165.73989 |
| x6:x17 | 2485.79314 |
| x6:x1 | −456.48044 |
| x7:x11 | 416.634369 |
| x7:x20 | 4347.85033 |
| x8:x10 | −764.12981 |
| x8:x17 | −9784.5653 |
| x10:x18 | −5012.6045 |
| x10:x20 | 7227.63801 |
| x13:x17 | −24491.605 |

The haematocrit estimates derived from the 21 and 23 electrode responses could thereby be used to correct the ketone measurements from electrode 24 for the expected biases that would result from the haematocrit sensitivity of that electrodes response.

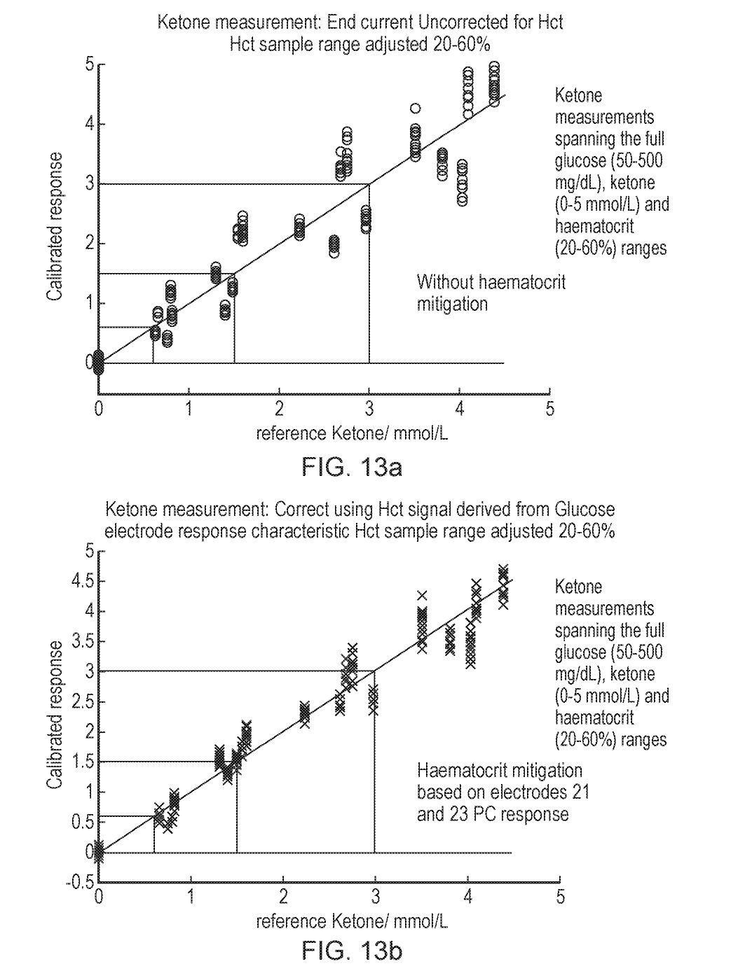

FIGS. 13a and 13b shows ketone measurement results for samples which spanned the entire glucose, haematocrit and ketone operating range. FIG. 13a shows ketone results which were based upon the end current calibration from FIGS. 12a-12c, and FIG. 13b shows measurement results which have been subsequently corrected for the expected haematocrit bias, based upon the haematocrit prediction from Algorithms 3a or 4a (depending upon the glucose level determined for the sample).

Example 2 thereby demonstrates how the problems of inaccuracy within a multi-analyte measurement system which arise through ‘crosstalk’ effects and haematocrit sensitivity can simultaneously be addressed without the need for increased sample volumes, with a test strip that is compatible with low cost high volume manufacturing methodology and which only requires a small sample volume that is comparable with existing state of the art single analyte SMBG systems. Such a test strip is furthermore simultaneously capable of measurement accuracy that is in line with or exceeding current regulatory requirements for single-analyte SMBG test strips.

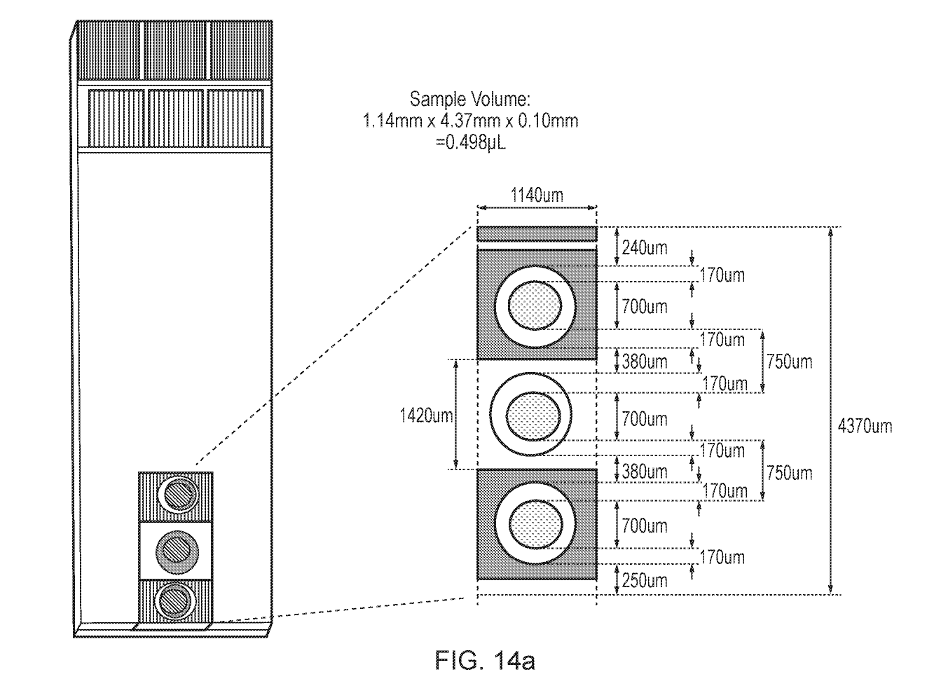

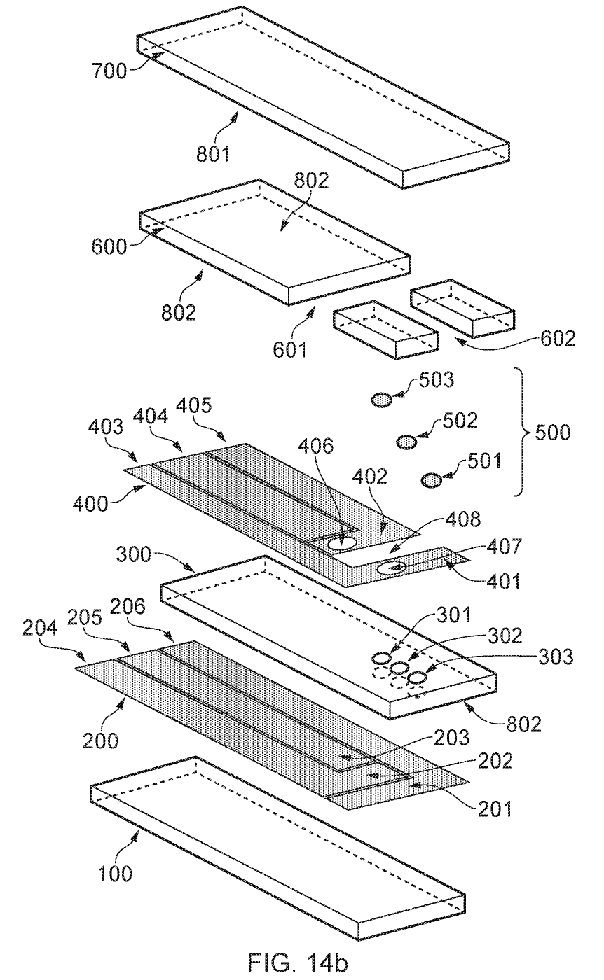

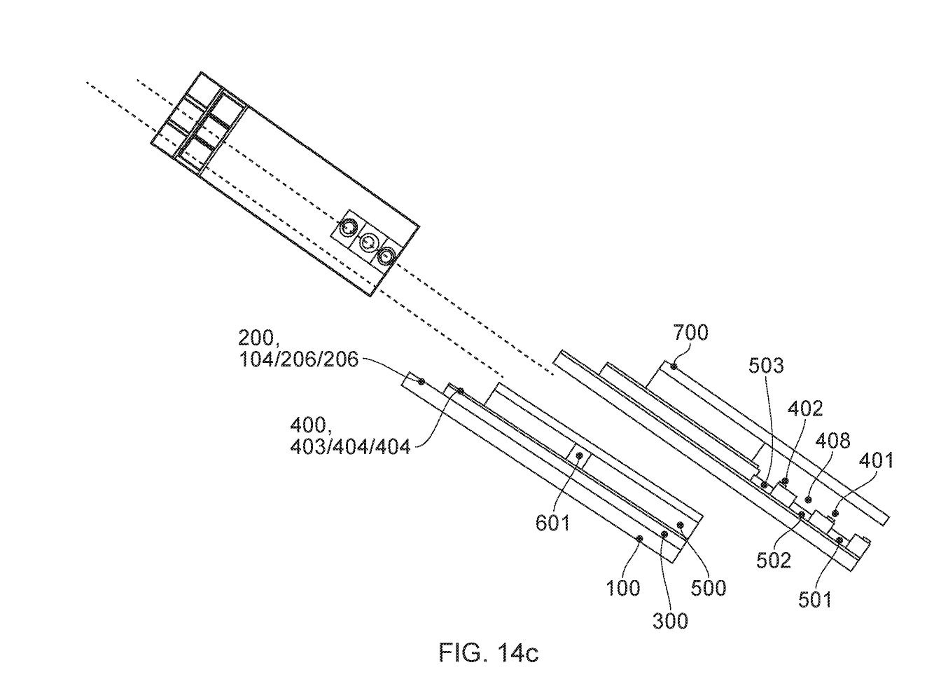

FIGS. 14a-14c show a further illustrative embodiment of the present invention. In particular, FIG. 14a shows a plan view of a low volume multi-analyte test strip. FIG. 14b shows an exploded, perspective view of the test strip. FIG. 14c shows the various features of the test strip of FIG. 14a in cross-section taken along an edge and centre line of the test strip when assembled.

With reference to FIGS. 14a-14c, the test device comprises a support layer or substrate (100) upon which a first conductive layer (200) is deposited. The conductive layer (200) comprises a plurality of working electrodes (201,203) and a reference/counter electrode (202), and corresponding electrode contacts (204,205,206). Above the first conductive layer (200) of patterned electrodes and fixed in place by means of a thin layer of adhesive (802) is a first dielectric spacer layer (300), within which there have been cut apertures (301,302,303) through which defined portions of the electrodes (201,202,203) are exposed. On top of the first dielectric spacer layer, a second conductive layer (400) comprised of a further plurality of electrodes (401,402), electrode contacts (403,405) an optional fill-sufficiency detect electrode (sliver) (404), annular void regions (406,407) surrounding both of the apertures (301,303), and a sample gap (408) over the middle aperture (302). Second conductive second layer 400 is arranged to provide error correction information for correcting a measurement of a response of a blood sample with at least one of reagent compositions (501,502,503) as will be described below.

A reagent layer (500) comprising multiple different reagent compositions (501,502,503) is deposited through the annular void regions (406,407) and the sample gap (408) and into the apertures (301,302,303) to cover the first layer of electrodes (201,202,203) with measurement reagent, whilst remaining clear of the second layer electrodes (401,402). A second dielectric spacer layer (600), within which a sample capillary space (602) and an air escape vent (601) are defined by appropriate voids, is disposed on top of the second conductive layer (400). The second dielectric spacer later (600) is held in place with a thin layer of adhesive (802). A second layer of adhesive (802) is used to fix a hydrophilic cover layer (700) which has been treated on its underside with a hydrophilic coating (701), onto the completed assembly.

FIG. 14a shows the typical dimensions of the main measurement elements within the sample capillary of the test strip and their respective positioning. The positioning outlined successfully achieves a required sample volume of <0.5 uL whilst optimally allowing manufacturing tolerances that are consistent with high speed, high volume, low cost manufacturing processes, and an arrangements of functional components that enables accurate measurements ostensibly free of crosstalk errors, while simultaneously enabling appropriate haematocrit mitigation strategies to be effected.

Specifically, in this embodiment the apertures (301,302,303) have a diameter of 700 microns (for example), and are separated by a gap of at least 720 microns from edge to edge at their closest approach. The reagent layers (501,502,503) are designed to sit within the reagent wells and substantially completely cover the exposed portions of the first layer electrodes (201,202,203). The annular void regions (406,407) extend beyond the limits of apertures (301,302,303) by an average distance of 170 μm thereby ensuring that the second layer of electrodes (401,402) are kept free from and sufficiently separated away from the reagent layers (501,502,503) which penetrate down into the apertures (301,302,303). The sample gap over the middle aperture is 1420 μm wide. Each of the electrodes of the second conductor layer (401,402) surrounds the annular void with a minimum dimension of about 90 μm at its thinnest point.

Approximately 100 μm of the (optional) 404 fill sufficiency detect electrode lies below the bottom edge of the air escape vent (601).

The capillary sample chamber 602 has a total width of 1140 μm and a total length of 4370 μm and an average height of 100 μm, producing a total volume of 0.498 μL. A small additional volume is required to allow for the depth of the apertures (301,302,303) over the electrodes; however this is offset by the fact that to operate the sample need only touch, not cover, the fill-sufficiency detect electrode (404).

In a non-limiting illustrative example the substrate 100 is a polyester substrate such as mylar, and the first conductive layer is a sputtered gold layer, with the electrode patterning accomplished by means of laser ablation.

The first dielectric layer 300 is a 20 μm laminate film and is sputtered with gold (400) and ablated by laser to define the electrode elements (401,402), the voids (406, 407) and the sample gap 408, and cut by laser to define the apertures (301,302,303), and coated on the underside with an adhesive 802.

The test device comprises a first analyte working electrode 201 where the analyte is beta-hydroxybutyrate (WE1K). The test device further comprises a second analyte working electrode 203 where the analyte is Glucose (WE2G). The test device further comprises a first impedance measurement electrode (I1) 401 and a second impedance measurement electrode (I2) 402.

Reagent layers 501,502,503 are deposited as micro-droplets by a ‘drop on demand’ system such as a Nordson Asymtek dispensing system.

### Illustrative Use of Embodiment

In a first illustrative use of the embodiment of FIGS. 14a-14c, the reagent layers (501, 502, 503) are preferred to undergo a relatively progressive dissolution and moderate to slow diffusion rate.

The contact end of the test strip is inserted into an appropriate meter and a potential is applied between the fill-sufficiency detect electrode 404 and the reference/counter electrode (202). A fluid sample is introduced to the capillary (602) and as soon as a resulting current is detected by the meter, the initial potential is switched off and the resistance of the fluid between electrodes (401) and (402) is measured, by AC impedance.

During this initial portion of the measurement, almost no redox species will yet have diffused from within the apertures (301,302) to reach impedance measurement electrodes (401) or (402), nor to penetrate the bulk of the blood sample located within the sample gap (408). An impedance measurement which is substantially free of reagent layer interference can be readily accomplished thereby. The sample gap (408) has adequate dimensions for a precise impedance measurement to be readily effected. This impedance measurement can thereafter be used to adjust the subsequent amperometric responses, and correct these for haematocrit by adjusting the measured responses according to the bias predicted to correspond to whichever haematocrit level corresponds to the impedance which was measured. The layout described optimally achieves an impedance measurement which is insensitive to the composition of the reagent layers, while simultaneously minimising the required sample volume. The layout is also compatible with the use of progressively (as opposed to rapidly) dissolving reagent layer compositions.

After a first time interval (during which the impedance measurement is completed) has elapsed, a voltage is then applied between the reference/counter electrode (202) and both of the analyte measurement electrodes (201 and 203). Amperometric measurements from each of the electrodes (201,203) are taken for a further time interval, which are then corrected according to the measured impedance.

In the event that a manufacturing defect or filling problem has led to misplacement or displacement of reagent from the first layer of electrodes, or where dissolution and/or diffusion is too rapid (and could therefore have interfered with the impedance measurement), this can be picked up by the second conductive layer of electrodes after the measurement has been completed and an error returned. This error detection can be effected by means of subsequent amperometric measurements between both electrodes 401 and 402 and the reference/counter electrode 202 at a suitable applied potential, wherein any higher than usual response can be used to signal an error state.

In this way, impedance signals from electrodes 401 and 402 can be used to correct for haematocrit. Amperometric response from electrodes 201 and 203 can be used to effect the underlying measurements, and amperometric responses from electrodes 401 and 402 used to detect errors that could result from crosstalk effects.

In an alternative illustrative use of the embodiment, one or more of the reagent layers (501, 502, 503) are preferred to undergo a relatively more rapid dissolution and a moderately rapid diffusion rate. Diffusion of electroactive reagents from each of the working electrode regions (201,203) to each of the second set of electrodes (401,402) can thereby be tracked by means of simultaneous or successive amperometric measurements of current at all of the (201,203,401,402) electrodes such that the differential response of the combined signals (201 with 401 and/or 203 with 402) is indicative of the rate at which mediator is diffusing through the sample. This allows for measurement of and correction for any crosstalk between 201 and 203 to be effected using the 401 and 402 (respectively) measurement signals.

Thus, in one embodiment a test strip may be provided which uses both: 1) analyte reagents with different diffusional and/or dissolutional characteristics (as per the embodiments of FIGS. 1a-1c and 7a-7c; and 2) a second conductive layer as per the embodiment of FIGS. 14a-14c. The first conductive layer comprising the working electrodes on which the analyte reagents are disposed, and the second conductive layer, would therefore both be arranged to provide error correction information for correcting a measurement of a response of a blood sample with at least one of the analyte reagents.

In yet a further alternative illustrative use of the embodiment, the overall rate of diffusion of electroactive species from the working electrode regions can be tracked using combined signals from electrodes (201, 202) compared against combined signals from electrodes (401, 402) combined, to give a response signal that is indicative of the rate of diffusion of all electroactive species in the system, which can then be used to derive a haematocrit correction term that can be applied to the raw measurement signal from electrode 201 and/or electrode 202.

In this way, amperometric responses from 201, 203, 401 and 402 can be combined to produce a haematocrit corrected response for each analyte without problems of crosstalk between the analytes.

Variations of the described embodiments are envisaged; for example, the features of all the disclosed embodiments may be combined in any way.

For example, an electrochemical test device may contain more layers than those disclosed in the preceding description. For example, an electrochemical test device may further comprise one or more bonding layers for bonding together one or more of the layers disclosed above. Additionally, some of the layers are not always necessary. For example, the test device may include a spacer layer that may define the interaction area of the electrodes of the conductor layer beneath. The spacer layer may perform the dual role of receiving a fluid sample through a capillary channel and defining an interaction area for combining the fluid sample with the conductor layer. For example, the spacer layer can, with appropriate adhesive, define the active area/interaction area of the electrodes.

In the examples of the electrochemical test device discussed above, a layer structure has been shown. The order in which each of the layers is formed may vary and any layer may, in some way, be configured so as to be in contact with any other layer.

The fluid sample may be a biological fluid. For example, the biological fluid may be blood, interstitial fluid, plasma, sweat, urine, lachrymal fluid, saliva or breath condensate. The one or more analytes may be any analyte(s) found in the fluid sample. For example, the analytes may be one or more of glucose, lactate, glycerol, cholesterol, free fatty acids, or a ketone body such as β-hydroxybutyrate, acetoactetate, acetone, β-hydroxy pentanoate, and β-keto pentanoate.

The electrochemical test strip may be configured to detect any combination of analytes so long as a suitable sensing chemistry is used. Example combinations include lactate and glycerol; lactate and β-hydroxybutyrate; lactate and glucose; glucose and glycerol; glucose and β-hydroxybutyrate; and glycerol and β-hydroxybutyrate to name a few. Further working electrodes may be provided allowing higher numbers of analytes to be measured. For example, the electrochemical test strip may be configured to detect glucose, glycerol and β-hydroxybutyrate; lactate, glycerol and glucose; or lactate, glycerol and β-hydroxybutyrate.

The electrochemical test device may be any suitable electrochemical test device. The electrochemical test device may be a test strip. In some examples the electrochemical test device may comprise a patch. Electrochemical test devices such as patches typically comprise a subcutaneous fluid extraction set and sensing chemistry for interaction with the one or more analytes. The electrochemical test device may be a monitoring component which transmits an output signal to a separate device such as a meter, either wirelessly or through a wired connection. The electrochemical test device may comprise a continuous monitoring device or a semi-continuous monitoring device.

In the examples discussed above, the electrochemical test devices have an end-fill configuration. In other embodiments, an electrochemical test device has a side-fill configuration i.e. the fluid sample is received at the side of the electrochemical test device.

In the examples provided above, the layers may be supplied using any suitable manufacturing technique. These include forms of printing, for example, screen printing, lithographic printing or tomographic printing. Other suitable manufacturing techniques include etching, and/or sputtering, chemical vapour deposition or physical vapour deposition.

A conductor layer may be formed of any suitable conductor. For example, the conductor layer may be formed from a carbon based paste, such as a carbon/graphite paste, including graphene. The conductor layer may be formed of one or more metal based paste such as a gold, platinum or silver paste. The electrodes may be formed of silver (Ag) or silver/silver chloride (Ag/AgCl). In some examples, the electrodes are formed of different conducting materials. The one or more working electrodes may, for example, be formed of carbon based ink whereas the counter/reference electrode may be formed of silver (Ag) or silver/silver chloride (Ag/AgCl).

The conductor layer may be of any suitable thickness. For example, the conductor layer may have a thickness greater than or equal to 0.005 mm and less than or equal to 0.030 mm.

The ordering of the electrodes on the electrochemical test device may be altered for efficiency. In one preferable option, an electrode for an analyte for which a weak signal is expected (for example glycerol or β-hydroxybutyrate which is often present in very low concentrations in blood) may be positioned closer to the entrance of the sample introduction chamber than an electrode for an analyte such as glucose or lactate which is usually present in higher concentrations.

The dielectric layer may be formed of any suitable insulating material. For example, dielectric/insulation inks may be polymer loaded inks that are thermoplastic, thermoset or UV cured and that, when dried or cured, form a contiguous non-conductive layer. Examples include, Loctite EDAG PF 021 E&C and DuPont 5018.

In the above-described embodiment of FIGS. 14a-14c, a polyester substrate layer was featured. Suitable substrate materials include polyester, polyimide, polystyrene, PVC, polycarbonate, glass and ceramic. When other layers are to be printed onto the substrate layer, the substrate layer has to be suitably printable for the chosen inks. The substrate must also be non-conductive. Typical thicknesses of the substrate layer range from 0.1 mm to 0.5 mm e.g. 0.35 mm. Glass and ceramic can be thicker as these are easier to handle with increased thickness. Thinner polymer substrates may be more difficult for the end user to use. Thicker substrates may offer some handling benefits.

The spacer layer may be formed of any suitable material. For example, the spacer layer may be made from a polyester core with a thin layer of PSA (Pressure Sensitive Adhesive) on either side. These adhesives can be the same or different depending on which layer is to be adhered to which side of the spacer layer.

Although in the examples above the thickness of the spacer layer was 0.1 mm, the thickness may vary. A typical range for the spacer layer thickness is 0.005-0.030 mm. Lower thicknesses may affect sensor performance and higher thicknesses would increase the volume of the sample introduction chamber. A thickness of an adhesive on the spacer layer may contribute to the rigidity of the spacer layer. Typically a spacer layer has a high volume resistivity. For example the volume resistivity may be greater than 1×10⁹ Ωcm. Other variations of the spacer layer are envisaged.

The sample introduction chamber may be provided along the longitudinal axis of the electrochemical device. The sample introduction chamber may be provided along the transverse axis of the electrochemical test device.

The vent may be of any suitable configuration for venting air from the sample introduction chamber. For example, the vent may comprise an air passageway in the cover. The vent may comprise an air passageway in the spacer layer. Optionally, air may be vented from the sample introduction chamber through one or more air passageways below the spacer layer, such as through the conductor layer or the dielectric layer.

Sensing chemistry may include any suitable mediator. In the examples described above, a ruthenium-based electron transfer agent has been disclosed for use with the second working electrode. Other suitable mediators may be osmium-based. For example, osmium phendione is a suitable mediator having a low standard redox potential. As another example, Os(4,4′-dimethyl-2,2′-bipyridine)₂ is a suitable mediator having a low standard redox potential.

An electron transfer agent for a working electrode may comprise a suitable quinone, for example a naphthoquinone derivative. The naphthoquinone derivative may be a 1,2 naphthoquinone derivative or a 1,4 naphthoquinone derivative. For example, the electron transfer agent may comprise 1,4 naphthoquinone-2-mercapto methyl carboxylic acid which has a standard redox potential of around −0.355V. The electron transfer agent may comprise 1,4 naphthoquinone-2-mercapto benzoic acid, which has a standard redox potential of around −0.345V. The electron transfer agent may comprise 1,2 naphthoquinone-4-sulphonate, which has a standard redox potential of around −0.214V. The electron transfer agent may comprise 1,4 naphthoquinone-2-mercapto methyl sulphonate. Also other suitable isomers of the above listed compounds are known which have similarly low standard redox potentials within the desired range.

In the above embodiments where an electron transfer agent is used, the electron transfer agent and the analyte reagent may be provided in the same layer or may be provided in different layers.

Whilst the above examples have been described primarily in the context of an electrochemical test device for measuring a concentration of an analyte in a bodily fluid, it may equally be used in other fields, for example in health and fitness, food, drink, bio-security applications and environmental sample monitoring. The examples described herein may equally be used in the context of animal/veterinary medicine and fitness (including dogs and horses).

The above embodiments have been described by way of example only, and the described embodiments are to be considered in all respects only as illustrative and not restrictive. It will be appreciated that variations of the described embodiments may be made without departing from the scope of the invention.

1. An electrochemical test device for detecting a first analyte and a second analyte in a fluid sample, the electrochemical test device comprising:
a first conductor layer arranged to receive a fluid sample, wherein the first conductor layer comprises a first working electrode for receiving sensing chemistry for the first analyte and a second working electrode for receiving sensing chemistry for the second analyte; and
wherein a layer of the electrochemical test device is arranged to provide error correction information for correcting a measurement of a response of the fluid sample with at least one sensing chemistry received on the first conductor layer.

2. An electrochemical test device according to claim 1,
wherein the first analyte is glucose and the second analyte is a ketone body, or wherein the first analyte is glucose and the second analyte is lactate, or wherein the first analyte is a ketone body and the second analyte is glycerol, or wherein the first analyte is lactate and the second analyte is glycerol,
optionally wherein the second analyte is β-hydroxybutyrate.

3. (canceled)

4. An electrochemical test device according claim 1, further comprising a spacer layer defining a sample introduction chamber for receiving input of the fluid sample into the electrochemical test device,
optionally wherein the sample introduction chamber has an upper volume of about 1.0 μL, optionally about 0.7 μL, further optionally about 0.45 μL, and still further optionally about 0.30 μL.

5. (canceled)

6. An electrochemical test device according to claim 1, wherein the first working electrode has thereon first sensing chemistry for the first analyte and the second working electrode has thereon second sensing chemistry for the second analyte.

7. An electrochemical test device according to claim 6,
wherein
the first sensing chemistry comprises a first analyte reagent formulated to react with the first analyte to generate a signal indicative of an amount of the first analyte in the fluid sample, the first analyte reagent having a first time-based response characteristic; and
wherein the sensing chemistry for the second analyte comprises a second analyte reagent formulated to react with the second analyte to generate a signal indicative of an amount of the second analyte in the fluid sample, the second analyte reagent having a second time-based response characteristic,
optionally wherein:
the first and second time-based response characteristics comprise respective
different diffusional and/or dissolutional response characteristics;
and/or
the first analyte reagent is formulated to more rapidly dissolve and/or diffuse
into the fluid sample relative to the second analyte reagent, optionally wherein:
the first and second analyte reagents comprise film-forming
components and non-film-forming components, the first analyte reagent comprising a relatively high proportion of non-film-forming components to film-forming components relative to the proportion of non-film-forming components to film-forming components comprised in the second analyte reagent.

8. (canceled)

9. (canceled)

10. (canceled)

11. An electrochemical test device according to claim 7, further comprising a third working electrode having thereon a third analyte reagent formulated to react with the first analyte to generate a signal indicative of an amount of the first analyte in the fluid sample, the third analyte reagent having a third time-based response characteristic,
optionally wherein the first analyte reagent is formulated to more rapidly dissolve and/or diffuse into the fluid sample relative to the third analyte reagent.

12. (canceled)

13. An electrochemical test device according to claim 1, further comprising a second conductor layer, wherein the second conductor layer is arranged to provide error correction information for correcting a measurement of a response of the fluid sample with at least one sensing chemistry received on the first conductor layer,
optionally wherein:
the second conductor layer comprises a first electrode and a second electrode, wherein the first electrode and the second electrode are spatially separated to form a pap arranged to receive a fluid sample;
and/or
the spacer layer is disposed between the first conductor layer and the second conductor layer.

14. (canceled)

15. (canceled)

16. An electrochemical test device according to claim 13, wherein the first electrode of the second conductor layer comprises a first through-hole and the second electrode of the second conductor layer comprises a second through-hole,
optionally wherein:
a first end of the first through-hole defines a first area on the first working electrode for receiving sensing chemistry for the first analyte;
and/or
a first end of the second through-hole defines a second area on the second working electrode for receiving sensing chemistry for the second analyte.

17. (canceled)

18. (canceled)

19. An electrochemical test device according to claim 16, further comprising an insulator layer disposed between the first conductor layer and the second conductor layer,
optionally wherein the insulator layer comprises a dielectric material.

20. (canceled)

21. An electrochemical test device according to claim 19, wherein the insulator layer comprises a third through-hole and a fourth through-hole,
optionally wherein the third through-hole fluidly connects the first through-hole to the first area and the fourth through-hole fluidly connects the second through-hole to the second area.

22. (canceled)

23. An electrochemical test device according to claim 16, wherein either:
the through-holes each have a diameter of about 700 μm;
and/or
the sample introduction chamber is fluidly-connected with the first through-hole and the second through-hole.

24. (canceled)

25. An electrochemical test device according to claim 21, wherein the internal volume of the sample introduction chamber, first, second, third and fourth through-holes is about 0.5 μl.

26. An electrochemical test device according to claim 4, further comprising a cover layer above the spacer layer for covering the top of the sample introduction chamber.

27. An electrochemical test device according to claim 1, wherein:
the fluid sample comprises one of: blood; plasma; urine; saliva; lacrimal fluid;
sweat; interstitial fluid; or breath condensate;
and/or
the first conductor layer further comprises a counter/reference electrode;
and/or
the sensing chemistry for the first analyte comprises a dehydrogenase,
optionally wherein:
the dehydrogenase is an NAD(P)⁺-dependent dehydrogenase and the sensing chemistry for the first analyte further comprises a cofactor for the NAD(P)⁺-dependent dehydrogenase,
optionally wherein:
the sensing chemistry for the first analyte further comprises a diaphorase and an electron transfer agent;
and/or
at least one of the sensing chemistry for the first analyte and the sensing chemistry for the second analyte comprises an oxidase.

28. (canceled)

29. (canceled)

30. (canceled)

31. (canceled)

32. (canceled)

33. An apparatus configured to detect a first analyte and a second analyte in a fluid sample applied to an electrochemical test device according to claim 1.

34. A method of making an electrochemical test device for measuring the amounts of first and second analytes in a fluid sample, comprising:
applying to a first electrode a first analyte reagent having a first time-based response characteristic and being formulated to react with a first analyte to generate a signal indicative of an amount of the first analyte in the fluid sample; and
applying to a second electrode a second analyte reagent having a second time-based response characteristic and being formulated to react with a second analyte to generate a signal indicative of an amount of the second analyte in the fluid sample.

35. A method of using an electrochemical test device according to claim 6, the method comprising:
polarising the first and second working electrodes;
applying a reference blood sample to the electrochemical test device, wherein the reference blood sample has known amounts of the first and second analyte and a known haematocrit; and
measuring one or more currents generated at each of the first and second working electrodes in the presence of the reference blood sample,
optionally further comprising:
reversing the polarisation of the first and second working electrodes; and
measuring one or more further currents generated at each of the first and second working electrodes in the presence of the reference blood sample;
and/or
determining, based on the one or more measured currents, sensitivity data indicative of a sensitivity of each of the first and second analyte reagents to: the first analyte, the second analyte and the haematocrit of the reference blood sample,
optionally further comprising:
determining additional data forming part of the sensitivity data by repeating said method for at least one further blood reference sample having known amounts of the first and second analyte and a known haematocrit,
optionally wherein:
each step of repeating for at least one further blood reference sample is performed on a respective at least one further electrochemical test device,
optionally wherein:
the electrochemical test device and at least one further electrochemical test device were manufactured in the same batch;
and/or
analysing the sensitivity data to determine a correction algorithm, wherein the correction algorithm compensates for the sensitivity of the first and second analyte reagents to haematocrit,
optionally further comprising:
applying a test blood sample to the test device;
measuring one or more currents generated at each of the first and second working electrodes;
correcting the one or more measured currents using the correction algorithm; and
determining an amount of the first analyte and an amount of the second analyte in the test blood sample, based on the corrected one or more measured currents.

36. (canceled)

37. (canceled)

38. (canceled)

39. (canceled)

40. (canceled)

41. (canceled)

42. (canceled)

43. A method of manufacturing an electrochemical test device, the method comprising:
providing a first conductor layer, wherein the first conductor layer comprises a first working electrode for receiving sensing chemistry for the first analyte and a second working electrode for receiving sensing chemistry for the second analyte; and
providing a layer arranged to provide error correction information for correcting a measurement of a response of the fluid sample with at least one sensing chemistry received on the first conductor layer.

44. A method according to claim 43, further comprising:
providing an insulator layer between the first conductor layer and the second conductor layer;
and/or
providing a spacer layer between the first conductor layer and the second conductor layer, the spacer layer defining a sample introduction chamber for introducing a fluid sample to the working electrodes.

45. (canceled)

46. A method of using an electrochemical test device according to claim 13, to determine an amount of a first analyte and an amount of a second analyte in a fluid sample, the method comprising:
applying an input signal to the second conductor layer;
measuring an impedance of the fluid sample to determine a correction algorithm;
applying an input signal to the first conductor layer to generate one or more output signals from the fluid sample;
correcting the one or more output signals using the correction algorithm;
determining an amount of the first analyte and an amount of the second analyte in the fluid sample, based on the corrected one or more output signals.
